SEQUENCE LISTING (14 sequences)

SEQ ID NO: 1 - DNA, 34 bp, Artificial Sequence
  Description of Artificial Sequence; note=synthetic construct
ataacttcgtataatgtatgctatacgaagttat

SEQ ID NO: 2 - DNA, 19 bp, Artificial Sequence
  Description of Artificial Sequence; note=synthetic construct
cttaccgtaacttgaaagt

SEQ ID NO: 3 - DNA, 14 bp, Artificial Sequence
  Description of Artificial Sequence; note=synthetic construct
ttttttcgtttttt

SEQ ID NO: 4 - DNA, 352 bp, Artificial Sequence
  Description of Artificial Sequence; note=synthetic construct
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatataacttcgtataatgtatgctatacgaagttatccgttttttcgttttttctccagcccgggaagatctataacttcgtataatgtatgctatacgaagttatccggcccattcctcctcggatcc

SEQ ID NO: 5 - DNA, 369 bp, Artificial Sequence
  Description of Artificial Sequence; note=synthetic construct
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcataacttcgtatatatatatctatacgaagttatgaaacaccgttttttcgttttttctccagcccgggaagatctataacttcgtatatatatatctatacgaagttatgaaacaccggcccattcctcctcggatccaagggtgggcgcgccgacccagc

SEQ ID NO: 6 - DNA, 365 bp, Artificial Sequence
  Description of Artificial Sequence; note=synthetic construct
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgattataacttcgtatatagtatgctatacgaagttatcaccgttttttcgttttttctccagcccgggaagatctataacttcgtatatagtatgctatacgaagttatcaccggcccattcctcctcggatccaagggtgggcgcgccgacccagc

SEQ ID NO: 7 - DNA, 370 bp, Artificial Sequence
  Description of Artificial Sequence; note=synthetic construct
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttataacttcgtatagtatatattatacgaagttatggaaacaccgttttttcgttttttctccagcccgggaagatctataacttcgtatagtatatattatacgaagttatggaaacaccggcccattcctcctcggatccaagggtgggcgcgccgacccagc

SEQ ID NO: 8 - DNA, 369 bp, Artificial Sequence
  Description of Artificial Sequence; note=synthetic construct
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttctaccgttcgtatatatatatctatacgaagttatgaaacaccgttttttcgttttttctccagcccgggaagatctataacttcgtatatatatatctatacgaacggtagaaacaccggcccattcctcctcggatccaagggtgggcgcgccgacccagc

SEQ ID NO: 9 - DNA, 464 bp, Artificial Sequence
  Description of Artificial Sequence; note=synthetic construct
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagtttttaaaattatgttttaaaatggactatcatatgcttaccgtaacttgaaagtatttcgatttcttggctttatatatcttgtggaaaggacgaaacaccgtgctcgcttcggcagcacatatactaaaattggaacgatacagagaagattagcatggcccctgcgcaaggatgacacgcaaattcgtgaagcgttccatatttttacatcaggttgtttttctgtttttacatcaggttgtttttctgtttggttttttttttacaccacgtttatacgccggtgcacggtttacca

SEQ ID NO: 10 - DNA, 707 bp, Artificial Sequence
  Description of Artificial Sequence; note=synthetic construct
gatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgtgctcgcttcggcagcacatatactaaaattggaacgatacagagaagattagcatggcccctgcgcaaggatgacacgcaaattcgtgaagcgttccatattttgttcctcagaggaactgacaagcaccctaacatcctattggaggctcactcacgttttttctattttgtttcttgacagcagagctcgttgctcactgtatagctcaggttggcctgacactgatgaggttctccagtgactgcctctacctacctactgggatgacagaggtgtaccaccaagccacgggctcctgtgtgagtgtgtgtgtgtgtgtataagtgtgccttccacagtgcacgtaagaggacaaggagttggttcttgctctcagatcatcaagct

SEQ ID NO: 11 - DNA, 523 bp, Artificial Sequence
  Description of Artificial Sequence; note=synthetic construct
tttaaacttagaacgaagcgagtataaaaaggattatttaaccctaaaacggattcaggatttgttataatatcaagtacagtcggctacataaggtcaccacatgtgtaaagttacaaaattctatggccttatatacctaccaagagcctgagtactctcggatgtgagggcgatctggctgcgacatctgtcaccccattgatcgccagggttgattcggctgatctggctggctaggcgggtgtccccttcctccctcaccgctccatgtgcgtccctcccgaagctgcgcgctcggtcgaagaggacgaccttccccgaatagaggaggaccggtcttcggtcaagggtatacgagtagctgcgctcctctgctagaacctccaaacaagctctcaaggtccattgtaggagaacgtagggtagtcaagcttccaagactccagacacatccaaatgaggcgctgcatgtggcagtctgctttcttttgtagttcctgcaatttaattttcgtttaaa

SEQ ID NO: 12 - DNA, 497 bp, Artificial Sequence
  Description of Artificial Sequence; note=synthetic construct
atttgcatgtcgctatgtgttctgggaaatcaccataaacgtgaaatgtctttggatttgggaatcttataagttctgtatgagaccactctttcccatagggcggagggaagctcatcagtggggccacgagctgagtgcgtcctgtcactccactcccatgtcccttgggaaggtctgagactagggccagaggcggccctaacagggctctccctgagcttcagggaggtgagttcccagagaacggggctccgcgcgaggtcagactgggcaggagatgccgtggaccccgcccttcggggaggggcccggcggatgcctcctttgccggagcttggaacagactcacggccagcgaagtgagttcaatggctgaggtgaggtaccccgcaggggacctcataacccaattcagaccactctcctccgcccatttttggaaaaaaaaaaaaaaaaaaaaaacaaaacgaaaccgggccgggcgcggtggttca

SEQ ID NO: 13 - DNA, 266 bp, Artificial Sequence
  Description of Artificial Sequence; note=synthetic construct
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagtttttaaaattatgttttaaaatggactatcatatgcttaccgtaacttgaaagtatttcgatttcttggctttatatatcttgtggaaaggacgaaacaccg

SEQ ID NO: 14 - DNA, 374 bp, Artificial Sequence
  Description of Artificial Sequence; note=synthetic construct
ttatagggagctgaagggaagggggtcacagtaggtggcatcgttcctttctgactgcccgccccccgcatgccgtcccgcgatattgagctccgaacctctcgccctgccgccgccggtgctccgtcgccgccgcgccgccatggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttccc

(19) United States
(12) Patent Application Publication
(10) Pub. No.: US 2006/0122381 A1
(43) Pub. Date: Jun. 8, 2006

(54) Recombinase mediated transcription

(76) Inventors: Jeffrey W. Streb, Oxnard, CA (US); Joseph M. Miano, Rochester, NY (US)

Correspondence Address: NEEDLE & ROSENBERG, P.C., SUITE 1000, 999 PEACHTREE STREET, ATLANTA, GA, 30309-3915, US

(21) Appl. No.: 10/533,976

(22) PCT Filed: Nov. 7, 2003
(86) PCT No.: PCT/US03/35645

§ 371 Date: Dec. 5, 2005

Related U.S. Application Data
(60) Provisional application No. 60/425,111, filed on Nov. 7, 2002.

(51) Int. Cl. A01K 67/027; C07H 21/04; C12N 15/74; C12N 5/06

(52) U.S. Cl. 536/23.7; 435/252.3; 435/471; 800/14; 435/325

(57) ABSTRACT

Disclosed are compositions and methods related to nucleic acids.

This application claims benefit of U.S. Provisional application No. 60/425,111, filed on Nov. 7, 2002, and which is herein incorporated by reference in its entirety.

### I. BACKGROUND

Transcription, the process of synthesizing RNA occurs in both Eukaryotic and prokaryotic cells. Typically, transcription is initiated at transcription start sites. Recombinant biotechnology has taken advantage of the transcription systems utilize by cells to produce vectors which carry a particular nucleic acid encoding for a desired product, which is typically not native to the cell to which it is going to be delivered, and then these vectors are delivered to any desired cell. It is of great interest to be able to control the expression of the encoded nucleic acid of the vector. Disclosed are compositions and methods for controlling the expression of nucleic acids.

### II. SUMMARY

Disclosed are methods and compositions related to nucleic acids for expression of transgenes.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows a schematic of a Recombinase-regulated RNA polymerase III dependent promoter (such as a Cre Recombinase-regulated U6 promoter) for restricted expression of small RNAs such as dsRNA hairpins. Insertion of a decoy, such as a LacZ reporter, flanked by LoxP sites separates the U6 promoter from its target, the hairpin. In the presence of Cre Recombinase, the intervening decoy is excised and the U6 promoter is brought into proximity of its target. Expression of the target hairpin is achieved only following recombination, allowing for indirect regulation of expression by regulating Cre recombinase expression or activity.

FIG. 2 shows a schematic of a promoter-recombinase nucleic acid.

FIG. 3 shows a bar graph indicating that insertion of recombinase recognition sequences, such as loxP, into the U6 promoter has minimal effect, as measured by the ability of a RNA hairpin expressed from these promoters to suppress luciferase expression.

FIG. 4 shows a schematic of constructs used to test recombination of U6-loxP constructs. Arrows indicate orientation of each component relative to each other. Note that the CMV promoter and Renilla gene are on the opposite strand relative to the U6 promoter and hairpin cassette.

FIG. 5 shows the results of the constructs set forth in FIG. 4.

FIG. 6 shows the results of constructs directed to exon 3 of the AKAPβ gene.

FIG. 7 shows that Cre recombinase can interfere with the ability of U6 promoters containing loxP sites. U6-loxP-Luc hairpin constructs were tested for their ability to silence expression of transiently over-expressed luciferase in the absence or presence of Cre recombinase. In all cases, the U6-loxP-Luc hairpin significantly inhibited luciferase expression in the absence of Cre recombinase, while the presence of Cre recombinase limited this effect. Thus, to achieve maximal function of the U6-loxP promoters, one can reduce either the expression or function of Cre recombinase following recombination.

FIG. 7 shows the organization of pol I transcription units. FIG. 7A shows that rRNA coding units are separated by intergenic spacers (IGS). FIG. 7B shows that the IGS contains a series of terminators (term), enhancers, a spacer promoter (SP), a proximal terminator (PT), the upstream promoter element (UPE) and the promoter core, which includes the rInr. The sites of transcription initiation are indicated by tis and/or the bent arrows.

FIG. 8 shows the organization of the three general pol III promoter types. The site of transcription initiation is indicated by +1 and the site of termination is indicated by “Term”. Also shown are the positions of various promoter elements, including the intermediate element (IE), proximal sequence element (PSE) and distal sequence element (DSE).

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS AND METHODS

There are many promoters that are constitutively active. The constitutive nature of these promoters is often desirable because of high expression of the desired product. However, this constitutive expression can also cause problems because of the inability of the promoters to be controlled, for example temporally or spatially. The standard way to address this problem is to use inducible promoters which are, for example, tissue specific or can be inducible by the presence of a particular reagent. The inducible systems have the drawback that to be active the inducible reagent must always be present or various desirable constitutive promoters cannot be used, such as RNA pol III promoters. Disclosed are compositions and methods which allow for the selective expression of constitutive promoters. It is understood that while the benefits of the disclosed promoters occur with constitutive promoters because particular problems arising from constitutive promoters are addressed, the disclosed compositions and methods can also be used with inducible or tissues specific promoters as well. Thus, any type of promoter, constitutive or otherwise can be used, but the problem of poorly being able to regulate expression from constitutive promoters is solved when the promoter is a constitutive promoter.

RNA polymerase III promoters are generally constitutively active. Disclosed are compositions and methods for preventing expression from a pol III promoter. The disclosed compositions can also be used with promoters of RNA pol I and II as well. The disclosed compositions and methods can involve separating the promoter from its transcribed sequence by insertion of a decoy spacer sequence. The disclosed compositions and methods can utilize existing recombination systems, such as the Cre recombinase system, to regulate the presence of the decoy spacer, which in turn regulates the positioning of the promoter from the transcribed sequence start site. That is, in the absence of recombination, the promoter and its target transcribed sequence are separated. Following recombination, however, the decoy spacer is removed and the promoter and its transcribed sequence are now adjacent to each other, allowing for expression of the transcribed sequence. By regulating expression and/or activity of a recombinase such as Cre, the recombination event required for expression from the RNA polymerase III promoter can be tightly controlled. This scheme for regulation is applicable in theory to all RNA polymerase III promoters, including the U6 promoter presented here, and is also applicable to all RNA polymerase II and I promoters. The disclosed promoters should be able to have recombinase recognition sequence(s) inserted, such as the LoxP sequence, into the promoter near the start site of transcription without significantly disrupting the promoter's activity.

### C. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular promoter is disclosed and discussed and a number of modifications that can be made to a number of molecules including the promoter are discussed, specifically contemplated are each and every combination and permutation of promoter and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

1. Recombinase Expression Construct

Disclosed are nucleic acids comprising a promoter and a set of recombinase sites, comprising a first recombination site and a second recombination site, wherein the promoter and the set of recombinase sites are oriented so that transcription from a transcription start site is increased when a recombinase acts on the set of recombinase sites after the nucleic acid is attached to a second nucleic acid forming a third nucleic acid and wherein the third nucleic acid comprises a transcription start site.

a) Decoy

The nucleic acids can have a variety of different types of sequences. For example the molecules typically have a decoy sequence which is the sequence in between two recombination sites. The decoy sequence is the sequence which will be removed when a recombinase acts on the recombination sequence. The decoy sequence can comprise different types of functional sequence. For example, the decoy sequence can comprise a marker sequence. The marker sequence can be any type of marker sequence and typically will be a sequence that allows for some type of selection of the nucleic acid, such as positive selection or negative selection. The selection can be based for example on viability of the cell or organism or the selection can be, for example visual, such as a color change (B-gal). The marker can be as described herein. The decoy can also comprise a transcription inhibitor recognition site. Such as a recognition site for a transcription repressor. The marker can be as described herein or for example, a marker conferring Neomycin Resistance, Zeocin Resistance, Blasticidin Resistance, Hygromycin Resistance or any other antibiotic resistance genes. Other markers could be Beta-Galactosidase, Green Fluorescent Protein and its variants, including EGFP, ECFP, EYFP, any other Fluorescent Proteins, including HcRed and DsRed, Cre Recombinase, FLP Recombinase, or other Lambda Integrases.

The decoy sequence can be any length that allows for the function of the disclosed nucleic acids, such as making a pol III promoter inducible.

b) Promoters

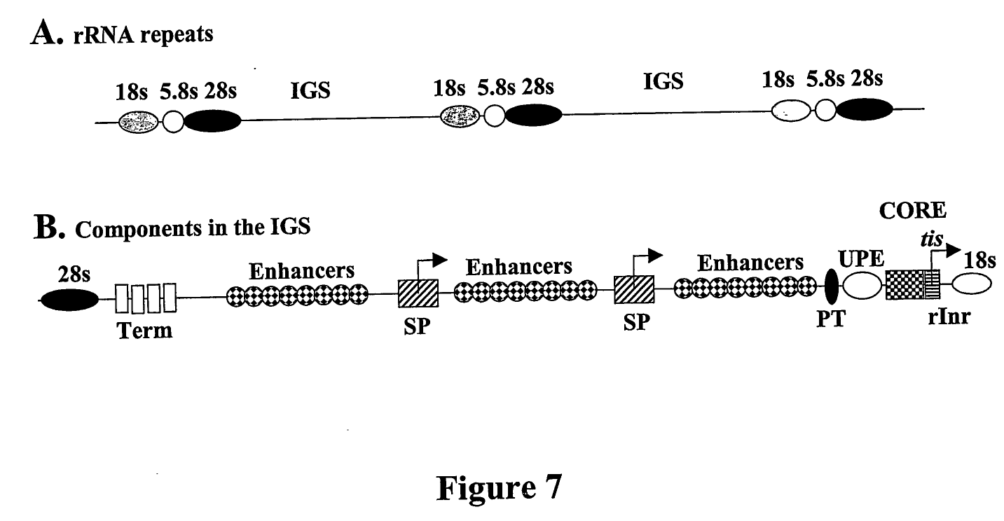

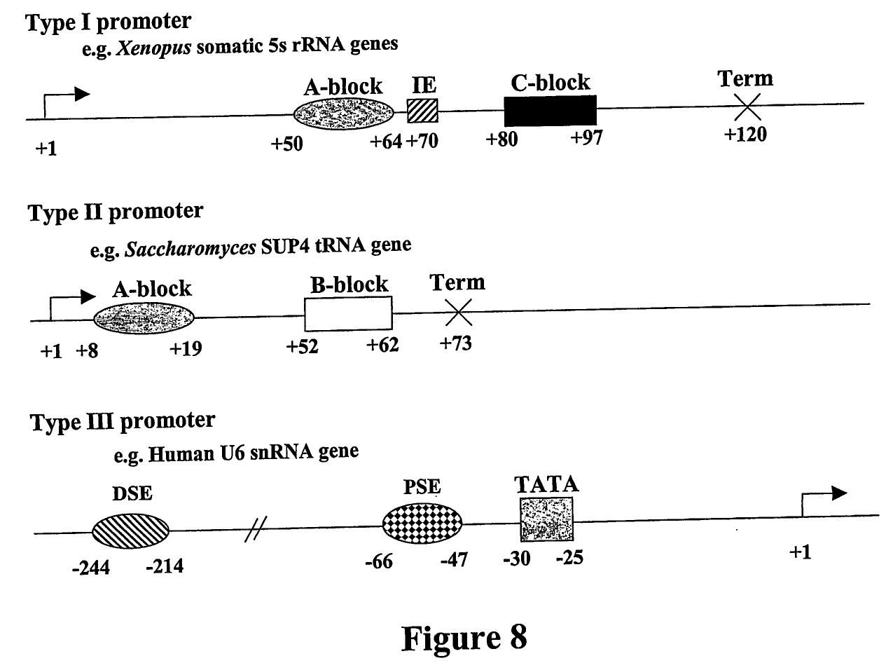

The nucleic acids also typically comprise a promotor. The promotor can be any type of promotor such as an RNA polymerase I (see FIG. 7) or II or III (see FIG. 8) promotor. The consensus sites and accepted variation of these promoters and their requirements are understood and are herein disclosed. Information about pol I promoters can be found in at least Reeder, R. H. (1984) Cell, 38, 349-351, Moss, T. and Stefanovsky, V. Y. (1994) Prog. Nucleic Acids Res. Mol. Biol., 50, 25-66; Paule, M. R. (1998) In Paule, M. R. (ed.), Transcription of Eukaryotic Ribosomal RNA Genes by RNA Polymerase I. Springer-Verlag, New York, N.Y., pp. 39-50; Planta, R. J. (1998) In Paule, M. R. (ed.), Transcription of Eukaryotic Ribosomal RNA Genes by RNA Polymerase I. Springer-Verlag, New York, N.Y., pp. 51-58; Grummt, I., et al., (1982) Nature, 296, 173-174; Pape, L. K, et al., (1990) Genes Dev., 4, 52-62; Muramatsu, M. (1998) In Paule, M. R. (ed.), Transcription of Eukaryotic Ribosomal RNA Genes by RNA Polymerase I. Springer-Verlag, New York, N.Y., pp. 295-308; and Marvin R. Paule and Robert J. White, “Transcription by RNA polymerases I and III,” Nucleic Acids Research, 2000, Vol. 28, No. 6 1283-1298, which are all herein incorporated by reference at least for material related to pol I promoters and their use.

(1) Pol III Promoters

Much information about Pol III promoters can be found in Nucleic Acids Research, Ying Huang and Richard J. Maraia, 2001, Comparison of the RNA polymerase III transcription machinery in Schizosaccharomyces pombe, Saccharomyces cerevisiae and human Nucleic Acids Res. Vol. 29, No. 13 2675-2690; Willis, I. M. (1993) “RNA polymerase III. Genes, factors and transcriptional specificity,” Eur. J. Biochem., 212, 1-11; Geiduschek, E. P. and Tocchini-Valentini, G. P. (1988) “Transcription by RNA polymerase III.” Annu. Rev. Biochem., 57, 873-914; Kassavetis, G. A., et al., (1994) In Conaway, R. C. and Conaway, J. W. (eds), Transcription: Mechanisms and Regulation. Raven Press, New York, N.Y., pp. 107-126; Chedin, S., et al., (1998) “The yeast RNA polymerase III transcription machinery: a paradigm for eukaryotic gene activation,” Cold Spring Harbor Symp. Quant. Biol., 63, 381-389; Paule, M. R. and White, R. J. (2000) Transcription by RNA polymerases I and III. Nucleic Acids Res., 28, 1283-1298; and Geiduschek, E. P. and Kassavetis, G. A. (1992) RNA polymerase III transcription complexes. In McKnight, S. L. and Yamamoto, K. R. (eds), Transcriptional Regulation. Cold Spring Harbor Laboratory Press, Cold Spring Harbor, N.Y., Vol. 1, pp. 247-280, all of which are herein incorporated by reference at least for material related to pol III transcription).\

Different RNA polymerase III promoters have different modes of TFIIIB recruitment.

On common characteristic of promoters used by pol III is that the majority require sequence elements downstream of +1, within the transcribed region. These internal control regions are generally discontinuous structures composed of essential blocks separated by non-essential regions.

Accurate and efficient termination as well as rapid re-initiation are important aspects of pol III transcription. Oligo dT acts as a termination signal in the non-template strand, but can also act to reinitiate transcription from the sense strand in S. cerevisiae and human (Dieci, G. and Sentenac, A. (1996) Facilitated recycling pathway for RNA polymerase III. Cell, 84, 245-252; Wang, Z. and Roeder, R. G. (1998) DNA topoisomerase I and PC4 can interact with human TFIIIC to promote both accurate termination and transcription reinitiation by RNA polymerase III. Mol. Cell, 1, 749-757). There are several factors that can stimulate or reinitiate transcription from pol III promoters including TFIIIC and associated components, TFIIIC1, TFIIIC0, topoisomerase I, positive factor 4 (PC4), a pol II co-activator, nuclear factor 1 (NF1) and the La antigen, a RNA UUU-OH-terminus-binding protein (Wang, Z. and Roeder, R. G. (1998) DNA topoisomerase I and PC4 can interact with human TFIIIC to promote both accurate termination and transcription reinitiation by RNA polymerase III. Mol. Cell, 1, 749-757; Oettel, S., et al., (1997) Human transcription factors IIIC2, IIIC1 and a novel component IIIC0 fulfill different aspects of DNA binding to various pol III genes. Nucleic Acids Res., 25, 2440-2447; Wang, Z., et al., (2000) Nuclear factor 1 (NF1) affects accurate termination and multiple-round transcription by human RNA polymerase III. EMBO J., 19, 6823-6832; Yoshinaga, S. K., et al., (1987) Resolution of human transcription factor TFIIIC into two functional components. Proc. Natl Acad. Sci. USA, 84, 3585-3589; Arrebola, R., et al., (1998) Tau91, an essential subunit of yeast transcription factor IIIC, cooperates with tau138 in DNA binding. Mol. Cell. Biol., 18, 1-9; Wang, Z. and Roeder, R. G. (1996) TFIIIC1 acts through a downstream region to stabilize TFIIIC2 binding to RNA polymerase III promoters. Mol. Cell. Biol., 16, 6841-6850; Fan, H., et al., (1997) Phosphorylation of the human La antigen on serine 366 can regulate recycling of RNA polymerase III transcription complexes. Cell, 88, 707-715; Gottlieb, E. and Steitz, J. A. (1989) Function of the mammalian La protein: evidence for its action in transcription termination by RNA polymerase III. EMBO J., 8, 851-861; Maraia, R. J., Kenan, D. J. and Keene, J. D. (1994) Eukaryotic transcription termination factor La mediates transcript release and facilitates reinitiation by RNA polymerase III. Mol. Cell. Biol., 14, 2147-2158; Goodier, J. L. and Maraia, R. J. (1998) Terminator-specific recycling of a B1-Alu transcription complex by RNA polymerase III is mediated by the RNA terminus-binding protein La. J. Biol. Chem., 273, 26110-26116; Lin-Marq, N. and Clarkson, S. G. (1998) Efficient synthesis, termination and release of RNA polymerase III transcripts in Xenopus extracts depleted of La protein. EMBO J., 17, 2033-2041; Weser, S., et al., (2000) Transcription efficiency of human polymerase III genes in vitro, does not depend on the RNP-forming autoantigen La. Nucleic Acids Res., 28, 3935-3942. all of which are incorporated by reference herein at least for material related to pol III transcription and initiation).

There are three subtypes of pol III promoters based on promoter structures and factor requirements. Type 1 are composed of a major internal element called the C box, and other elements that vary among species, examples of which are the promoters of 5S rRNA genes. (Geiduschek, E. P. and Kassavetis, G. A. (1992) RNA polymerase III transcription complexes. In McKnight, S. L. and Yamamoto, K. R. (eds), Transcriptional Regulation. Cold Spring Harbor Laboratory Press, Cold Spring Harbor, N.Y., Vol. 1, pp. 247-280, which is herein incorporated by reference at least for material related to pol III transcription).

(a) Type I

An example of a type I pol III promoter is the promoter of the somatic 5S rRNA gene in Xenopus laevis. This promoter requires three internal elements for efficient transcription: an A block located between +50 and +64, an intermediate element at +67 to +72 and a C block from +80 to +97 (Pieler, T., Hamm, J. and Roeder, R. G. (1987) Cell, 48, 91-100 herein incorporated by reference at least for material related to pol III promoters) This promoter is also used as an example and is shown in FIG. 8. This set up is also found in the 5S rRNA genes of other lower organisms, including Drosophila melanogaster (Sharp, S. J. and Garcia, A. D. (1988) Mol. Cell. Biol., 8, 1266-1274 herein incorporated by reference at least for material related to pol III promoters) and S. cerevisiae (Lee, Y., et al., (1995) Nucleic Acids Res., 23, 634-640 herein incorporated by reference at least for material related to pol III promoters), and is unique to the 5S rRNA genes.

(b) Type 2

Type 2 pol III promoters, the most common, can be found in tRNA genes, adenovirus VA genes, Alu sequences and other short interspersed elements. Type 2 promoters are also internal, but have two highly conserved sequence elements. The sequence most proximal to the start site is called the proximal A box (10-20 bp of start) and the more distal site is called the distal B box, both of which are within the transcribed region. The A regions are homologous between type 1 and type 2 and can be interchanged. (Ciliberto, G., et al., (1983) Cell, 32, 725-733, herein incorporated at least for material related to type 1 and type 2 promoters). For example, a Xenopus tRNA^{Leu} gene has its A block between +11 and +21, ˜40 bp further upstream than the A block of the Xenopus 5S rRNA genes (Galli, G., et al., (1981) Nature, 294, 626-631). The distances between the A and B boxes can vary, and can include different intron lengths. (Geiduschek, E. P. and Kassavetis, G. A. (1992) RNA polymerase III transcription complexes. In McKnight, S. L. and Yamamoto, K. R. (eds), Transcriptional Regulation. Cold Spring Harbor Laboratory Press, Cold Spring Harbor, N.Y., Vol. 1, pp. 247-280, which is herein incorporated by reference at least for material related to pol III transcription). The distances, can be for example, anywhere between 20 and 70 bases long, as well as anywhere between 30 and 60 bases long. The distances can be as long as 100, 200, 300, 365, 400, and 500 bases long. (Galli, G., et al., (1981) Nature, 294, 626-631, Baker, R. E., et al., (1987) Proc. Natl. Acad. Sci. USA, 84, 8768-8772 herein incorporated at least for material related to the length between the A and B boxes) The ability to accept many different distances comes in part by TFC4p, a large protein with multiple tetratricopeptide repeats (TPR) that appears to provide the flexibility required for such broad ranging promoter spacing situations. (Joazeiro, C. A., Kassavetis, G. A. and Geiduschek, E. P. (1996) Alternative outcomes in assembly of promoter complexes: the roles of TBP and a flexible linker in placing TFIIIB on tRNA genes. Genes Dev., 10, 725-739, which is herein incorporated by reference at least for material related to pol III transcription). In addition to the A and B box, there is another separate control element called the pol III terminator, which is typically located 20-25 bp downstream of the B box, within, for example, tRNA genes.

For type 1 and 2 promoters the proximal subunits of TFIIIC direct TFIIIB to bind upstream of the transcription start site (Kassavetis, G. A., et al., (1992) “The role of the TATA-binding protein in the assembly and function of the multisubunit yeast RNA polymerase III transcription factor, TFIIIB,” Cell, 71, 1055-1064; Hsieh, Y. J., et al., (1999) “Cloning and characterization of two evolutionarily conserved subunits (TFIIIC102 and TFIIIC63) of human TFIIIC and their involvement in functional interactions with TFIIIB and RNA polymerase III,” Mol. Cell. Biol., 19, 4944-4952 all of which are herein incorporated by reference at least for material related to pol III transcription). TFIIIB then recruits and positions pol III over the initiation site and remains stably bound to the DNA through multiple rounds of re-initiation by pol III (Kassavetis, G. A., et al., (1990) S. cerevisiae TFIIIB is the transcription initiation factor proper of RNA polymerase III, while TFIIIA and TFIIIC are assembly factors. Cell, 60, 235-245).

(c) Type 3

Type 3 pol III promoters (vertebrate) do not need intragenic promoter elements. Examples are the human and mouse U6 snRNA promoters which retain full activity following deletion of all sequences downstream of +1 (Das, G., et al., (1988) EMBO J., 7, 503-512; Kunkel, G. R. and Pederson, T. (1989) Nucleic Acids Res., 17, 7371-7379; Lobo, S. M. and Hernandez, N. (1989) Genes Dev., 58, 55-67 all herein incorporated at least for material related to U6 promoters). Other examples of this are the human 7SK and MRP/7-2 RNA genes. (Murphy, S., et al., (1987) Cell, 51, 81-87; Yuan, Y. and Reddy, R. (1991) Biochim. Biophys. Acta, 1089, 33-39 all herein incorporated at least for material related to U6 promoters). In yeast, U6 genes have functional A and B blocks, albeit in unusual positions (Brow, D. A. and Guthrie, C. (1990) Genes Dev., 4, 1345-1356) and a U6 gene with an entirely internal promoter has even been found in humans (Tichelaar, J. W., et al., (1994) Mol. Cell. Biol., 14, 5450-5457).

There are many different examples of type 3 genes which include the vertebrate U6, 7SK, hY4, hY5 and H1 snRNA genes. Type 3 promoters rely on an upstream TATA element that functions as one component of an entirely upstream multi-partite promoter (Hannon, G. J., et al., (1991) Multiple cis-acting elements are required for RNA polymerase III transcription of the gene encoding H1 RNA, the RNA component of human RNase P. J. Biol. Chem., 266, 22796-22799, Hernandez, N. (1992) Transcription of snRNA genes and related genes. In McKnight, S. L. and Yamamoto, K R. (eds), Transcriptional Regulation. Cold Spring Harbor Laboratory Press, Cold Spring Harbor, N.Y., pp. 281-313; Maraia, R. J., et al., (1996) Gene encoding human Ro-associated autoantigen hY5 RNA. Nucleic Acids Res., 24, 3552-3559; Maraia, R. J., et al., (1994) The human Y4 small cytoplasmic RNA gene is controlled by upstream elements and resides on chromosome 7 with all other hY scRNA genes. Nucleic Acids Res., 22, 3045-3052 al of which are incorporated by reference at least for material related to the type 3 pol III promoters and their elements).

The best characterized type III promoter belongs to a human U6 gene (FIG. 8). The U6 promoter uses a TATA box, between −30 and −25, a proximal sequence element (PSE) between −66 and −47 and a distal sequence element (DSE) between −244 and −214. The U6 PSE and DSE are homologous and interchangeable with elements found at comparable positions in the U2 snRNA gene transcribed by pol II, even tough a TATA box is not found in the U2 promoter; this is a curious anomaly, since TATA sequences are a classic feature of class II rather than class III genes. Even more paradoxical is the observation that inserting a TATA box can convert U2 into a pol III promoter, whereas crippling its TATA box allows U6 to be transcribed by pol II. Clearly, the U snRNA genes are a law unto themselves. The human U6 snRNA gene has an upstream TATA box, a proximal sequence element (PSE) and a distal sequence element (DSE). The PSE and TATA element work together to bring the Transcription Factors SNAPc/PTF and a TFIIIB-like activity (TFIIIB-α) to the promoter. (Hernandez, N. (1992) Transcription of snRNA genes and related genes. In McKnight, S. L. and Yamamoto, K. R. (eds), Transcriptional Regulation. Cold Spring Harbor Laboratory Press, Cold Spring Harbor, N.Y., pp. 281-313; Lobo, S. M. and Hernandez, N. T. (1994) Transcription of snRNA genes by RNA polymerases II and III. In Conaway, R. C. C. and Conaway, J. W. (eds), Transcription: Mechanisms and Regulation. Raven Press, New York, N.Y., pp. 127-159; Henry, R. W., et al., (1998) Crossing the line between RNA polymerases: transcription of human snRNA genes by RNA polymerases II and III. Cold Spring Harbor Symp. Quant. Biol., 63, 111-120; Yoon, J. B., et al., (1995) Proximal sequence element-binding transcription factor (PTF) is a multisubunit complex required for transcription of both RNA polymerase II- and RNA polymerase III-dependent small nuclear RNA genes. Mol. Cell. Biol., 15, 2019-2027 all of which are incorporated by reference at least for material related to transcription factors involved in pol III promoter function).

The transcriptional activator Oct-1 can be recruited by the upstream DSE (Lescure, A., et al., (1992) A factor with Sp1 DNA-binding specificity stimulates Xenopus U6 snRNA in vivo transcription by RNA polymerase III. J. Mol. Biol., 228, 387-394; Murphy, S., et al., (1992) Oct-1 and Oct-2 potentiate functional interactions of a transcription factor with the proximal sequence element of small nuclear RNA genes. Mol. Cell. Biol., 12, 3247-3261; Danzeiser, D. A., et al., (1993) Functional characterization of elements in a human U6 small nuclear RNA gene distal control region. Mol. Cell. Biol., 13, 4670-4678; Schaub, M., et al., (1997) Staf, a promiscuous activator for enhanced transcription by RNA polymerases II and III. EMBO J., 16, 173-181 all of which are incorporated for material at least related to pol III promoters and their activity) and can function in part by promoting binding of the SNAPc complex to the PSE (Mittal, V. and Hernandez, N. (1997) Role for the amino-terminal region of human TBP in U6 snRNA transcription. Science, 275, 1136-1140; Mittal, V., Ma, B. and Hernandez, N. (1999) SNAP(c): a core promoter factor with a built-in DNA-binding damper that is deactivated by the Oct-1 POU domain. Genes Dev., 13, 1807-1821 all of which are incorporated for material at least related to pol III promoters and their activity). A stable initiation complex is formed by cooperative interactions between TFIIIB, SNAPc and Oct-1 bound to their respective promoter elements, in part mediated by a nucleosome that is positioned between the DSE and PSE (Danzeiser, D. A., et al., (1993) Functional characterization of elements in a human U6 small nuclear RNA gene distal control region. Mol. Cell. Biol., 13, 4670-4678; Schaub, M., et al., (1997) Staf, a promiscuous activator for enhanced transcription by RNA polymerases II and II. EMBO J., 16, 173-181; Mittal, V., et al., (1999) SNAP(c): a core promoter factor with a built-in DNA-binding damper that is deactivated by the Oct-1 POU domain. Genes Dev., 13, 1807-1821 all of which are incorporated for material at least related to pol III promoters and their activity).

The U6 promoter of S. cerevisiae (sc) is different than the vertebrate U6 in that it does not have a PSE or DSE but does have a downstream B box, which is about 120 bases past the terminator. (Brow, D. A. and Guthrie, C. (1990) Transcription of a yeast U6 snRNA gene requires a polymerase III promoter element in a novel position. Genes Dev., 4, 1345-1356; Eschenlauer, J. B., et al., (1993) Architecture of a yeast U6 RNA gene promoter. Mol. Cell. Biol., 13, 3015-3026 all of which are incorporated herein for material at least related to issues related to sc U6 and vertebrate U6 promoters). Thus, the scU6 is not a type 1 or 2 pol III promoter because even though the scU6 snRNA gene (SNR6) uses TATA and B box elements these elements are external to the transcribed region. Furthermore, it is not exactly like a vertebrate type 3 gene because vertebrate type 3 promoters use TATA and other promoter elements that are entirely upstream.

(d) Non Type 1, 2, or 3

While many pol III promoters can be categorized as 1, 2, or 3 there are some that use elements of all three, and therefore are not easily categorized. However, a unifying factor between all pol III promoters is the recruitment of important pol III transcription factors such as PSE/SNAPc and B box/TFIIIC, and the recruitment of TFIIIB to the start site of transcription and to stabilize its presence. Often this includes interactions with the TATA-binding protein (TBP) of TFIIIB and a TATA promoter element, while in other cases TBP, with associated TFIIIB subunits, is induced to bind the upstream DNA in the absence of a TATA motif (Persinger, J., et al., (1999) Spatial organization of the core region of yeast TFIIIB-DNA complexes. Mol. Cell. Biol., 19, 528-5234 incorporated by reference herein for material at least related to transcription from pol III promoters).

An example is the EBER2 gene of Epstein-Barr virus has A and B blocks that are typical of type II promoters and are essential for transcription. But in this promoter, deletion of sequences upstream of −46 reduces expression in transfected cells to 7% of the wild-type level. It is believed that Upstream binding sites for Sp1 and ATF are thought to be responsible for this effect. Howe, J. G. and Shu, M.-D. (1989) Cell, 57, 825-834. The EBER2 promoter also has a TATA box between −28 and −23 that increases its activity 5-fold. Other examples include silkworm tRNA^{Ala} genes (Sprague, K. U., et al., (1980) Cell, 22,171-178), the Xenopus tRNA^{Sec} gene (Carbon, P. and Krol, A. (1991) EMBO J., 10, 599-606), the rat vault RNA gene (Vilalta, A., et al., (1994) J. Biol. Chem., 269, 29752-29759) and the human 7SL gene (Ullu, E. and Weiner, A. M. (1985) Nature, 318, 371-374).

c) Recombinase Systems

The recombinase sites can be sites that are utilized in any type of recombinase system. Recombinases generally rearrange nucleic acid, such as DNA. The recombinase sites can be recognized by lambda integrase, i.e. tyrosine integrases. Examples of lambda integrases are phage lambda integrase, bacteriophage P1 Cre recombinase, a XerC/XerD recombinases, and Flp recombinase. The recombinases sites can also be recognized by resolvase invertases. Examples of resolvase invertases are gamma-delta resolvase, TN3 transposon resolvase, Gin invertase, and Hin invertase.

The elements of the disclosed nucleic acids are arranged such that after a recombinase acts on the recombinase sites the transcription activity from the associated promotor increases at an associated start site. This effect can arise from a spatial orientation that separates the promoter from the transcription start site. This can arise from steric hinderance associated with molecules binding the decoy sequence or from transcription repressors being associated with the decoy sequence. For example, the promoter and the set of recombinase sites can be oriented 5′ to 3′ such that the promoter is followed by a first recombinase site which is then followed by a second recombinase site.

The promoter can be separated from the transcription start site by at least 50, 75, 100, 150, 200, 300, 400, 500, 600, 800, 1000, 1500, 2000, 2500, 3000, 4000, or 5000 nucleotides.

It is understood that the promotor sites and recombinase sites can be in any orientation such that the recombinase sites reduce the effect the promoter has on a given transcription start site.

d) Inducible and Tissue Specific Recombinase Systems

It is understood that disclosed are inducible promoter systems for, for example, pol III promoters. In certain situations nucleic acids as disclosed herein can be used to produce, for example, cells that can then have the pol III promoter induced can be produced. In the presence of a recombinase, the disclosed cells, would turn on the expression of the pol III promoter. In certain embodiments the mice or other transgenic animals having the promoters can be produced. In those cases, a mouse, for example, having the floxed promoter can be made and then crossed with a mouse that for example produces a constitutive cre in all cells under, for example, the beta actin promoter, in other systems, the it could be a tissue specific promoter driven cre. It is understood that the recombinant systems can be used in an inducible system. Furthermore, it is understood that the recombinant systems can be expressed in a tissue-specific manner. Disclosed are methods wherein the cre recombinase is under the control of the EIIA promoter, a promoter specific for breast tissue, such as the WAP promoter, a promoter specific for ovarian tissue, such as the ACTB promoter, or a promoter specific for bone tissue, such as osteocalcin. Any tissues specific promoter can be used. Promoters specific for prostate, testis, and neural are also disclosed. Examples of some tissue-specific promoters include but are not limited to MUC1, EIIA, ACTB, WAP, bHLH-EC2, HOXA-1, AFP, opsin, CR1/2, Fc-γ-Receptor 1 (Fc-γ-R1), MMTVD-LTR, the human insulin promoter, Pdha-2, rat neuron-specific enolase,

Disclosed are inducible expression systems to generate mice with knockouts caused by functional nucleic acids, for example, such as an RNAi situation. It is understood that many inducible expression systems exist in the art and may be used as disclosed herein. Inducible expression systems can include, but are not limited to the Cre-lox system, Flp recombinase, HIN recombinase, Rad52 recombinase, XerD recombinase, RecA recombinase, Mpi recombinase, and tetracycline responsive promoters. The Cre recombinase system which when used will execute a site-specific recombination event at loxP sites. A segment of DNA that is flanked by the loxP sites, floxed, is excised from the transcript.

To create null mice using the Cre-lox system, two types of transgenic mice are created. The first is a mouse transgenic for Cre recombinase under control of a known inducible and/or tissue-specific promoter. The second is a mouse that contains the floxed promoters disclosed herein. These two transgenic mouse strains are then crossed to create one strain comprising both mutations. Control of the recombination event, via the Cre Recombinase, can be constitutive or inducible, as well as ubiquitous or tissue specific, depending on the promoter used to control Cre expression. Disclosed is a constitutive system in which the Cre recombinase is expressed from a β-actin promoter. Other inducible expression systems exist and can be used as disclosed herein. Disclosed herein, a non-tissue specific promoter, β-actin, is used in the form of the FVB/N-TgN(ACTB-Cre)2Mrt (stock # 003376) mice (Jackson Laboratory, Bar Harbor, Me.). However, the CMV promoter and adenovirus EIIa promoter, for example, are also examples of ubiquitous promoters and can be substituted for β-actin to achieve the same result. Also disclosed are constructs and their use comprising the WAP promoter for the establishment of breast specific induction. B6129-TgN(WAPCre)1738Mam (stock # 003552) (Jackson Laboratory, Bar Harbor, Me.) mice can be used to establish tissue-specific Cre recombinase expression, with Cre under the control of WAP. It is understood that other expression systems may be substituted for the Cre expression system disclosed herein. It is anticipated that variations in the expression system used can result in a need to change other components of the recombination event, for example, the promoter. Commercially available mice (Jackson Laboratory, Bar Harbor, Me.) that utilize the cre-lox inducible expression system include at least 129-TgN(PRM-Cre)58Og (stock # 003328), 129.Cg-Foxg1^{tm1(Cre)Skm} (stock # 004337), 129S6-Tg(Prnp-GFP/Cre) 1 Blw (stock #003960), B6.129-Tg(Pcp2-Cre)2 Mpin (stock # 004146), B6.129S4-Meox2^{CreSor} (stock # 003755), B6.Cg(D2)-TgN (xstpxLacZ)32And (stock # 002982), B6.Cg(SJL)-TgN(NesCre)1Kln (stock # 003771), B6.Cg-Tg(Rbp3-Cre)528Jxm (stock # 003967), B6.Cg-Tg(Syn1-Cre)671Jxm (stock # 003966), B6.Cg-Tg(Tek-Cre)12Flv (stock # 004128), B6.Cg-TgN(LckCre)548Jxm (stock # 003802), B6.FVB-TgN(EIIa-Cre)C5379Lmgd (stock # 003724), B6129-TgN(MMTV-Cre)1Mam (stock # 003551), B6129-TgN(MMTV-Cre)4Mam (stock # 003553), B6129-TgN(WAPCre)11738Mam (stock # 003552), B6;D2-TgN(Sycp1-Cre)4 Min (stock # 003466), B6;FVB-TgN(GZMB-Cre)1Jcb (stock # 003734), B6;SJL-TgN(Col2a1-Cre)1Bhr (stock # 003554), BALB/c-TgN(CMV-Cre)#Cgn (stock # 003465), C.129P2-Cd19^{tm1(Cre)Cgn} (stock # 004126), C57BL/6-TgN(AlbCre)21Mgn (stock # 003574), C57BL/6-TgN(Ins2Cre)25Mgn (stock # 003573), C57BL/6-TgN(Zp3-Cre)3Mrt (stock # 003394), C57BL/6-TgN(Zp3-Cre)93Knw (stock # 003651), C57BL/6-TgN(Mx1-Cre)1Cgn (stock # 003556), DBA/2, TgN(xstpxLacZ)36And (stock # 002981), FVB/N-TgN(ACTB-Cre)2Mrt (stock # 003376), FVB/N-TgN(EIIa-Cre)C5379Lmgd (stock # 003314), FVB/N-TgN(Zp3-Cre)3Mrt (stock # 003377), STOCK Mttp^{tm1Sgy}Ldlr^{tm1Sgy}Apob^{tm1Sgy}Tg(Mx-Cre)1Cgn (stock # 004192), STOCK TgN(Wnt1-GAL4)11Rth (stock # 003829), STOCK TgN(Wnt1-Cre)11Rth (stock # 003829), STOCK TgN(balancer1)2Cgn (stock # 002858), STOCK TgN(balancer2)1Cgn (stock # 002859), and STOCK TgN(hCMV-Cre)140Sau (stock # 002471). Among these mice, B6.Cg(SJL)-TgN(NesCre)1Kln (stock # 003771), B6.Cg-Tg(Syn1-Cre)671Jxm (stock # 003966), and C57BL/6-TgN(Ins2Cre)25Mgn (stock # 003573) are examples of mice that have tissue specific Cre promoters. The B6.Cg-TgN(LckCre)548Jxm (stock # 003802) mice place Cre under control of the Lck promoter and do not have tissue specificity. The B6.FVB-TgN(EIIa-Cre)C5379Lmgd (stock # 003724) and BALB/c-TgN(CMV-Cre)#Cgn (stock # 003465) also have Cre recombinase under the control of a non-tissue-specific promoter. The disclosed floxed promoter mice may be crossed with any of the Cre mice available to take advantage of additional promoter activity and specificity. Commercially available mice (Jackson Laboratory, Bar Harbor, Me.) that utilize the Flp recombinase expression system are 129S4/SvJaeSor-Gt(ROSA)26Sor^{tm1(FLP1)Dym} (stock # 003946) and B6;SJL-TgN(ACTFLPe)9205Dym (stock # 003800). Also disclosed are the Offspring of the disclosed floxed promoter mice crossed with the disclosed Cre mice.

e) Transgenes

The disclosed nucleic acids can also comprise an associated transgene whose transription is repressed by the associated recombinase sites and is increased after a recombinase acts on the recombinase sites. The transgene can be any desired expressed nucleic acid. For example, the transgene can be an expressed mRNA leading to an expressed polypeptide. The transgene could also express some type of functional nucleic acid such as an RNAi or a ribozymes. The transgene, can also reside on a target nucleic acid that will be reacted with the nucleic acids comprising the recombinase sites and promoters, for example. Different types of transgenes can be expressed, and in certain situations, the transgenes themselves can be floxed, such that the transgene will not function, before recombination, but will function after recombination. For example, the transgene could be an siRNA that is floxed, such that the sense and antisense strands do not come together until recombination occurs. (see Kasim et al., Nucleic Acid Research Supplement, 3:255-256 (2003), which is herein incorporated at least for material related to siRNA and its control and recombination).

As discussed above, the transgene can be a variety of different types of sequence including sequence containing a functional nucleic acid.

(1) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affecters, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA or the genomic DNA or they can interact with a particular polypeptide. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶, 10^{-8, 10}⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. For example, when determining the specificity of aptamers, the background protein could be serum albumin. Representative examples of how to make and use aptamers to bind a variety of different target molecules can be found in the following non-limiting list of U.S. Pat. Nos. 5,476,766, 5,503,978, 5,631,146, 5,731,424 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukaryotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al. Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

It is also understood that the disclosed nucleic acids can be used for RNAi or RNA interference. It is thought that RNAi involves a two-step mechanism for RNA interference (RNAi): an initiation step and an effector step. For example, in the first step, input double-stranded (ds) RNA (siRNA) is processed into small fragments, such as 21-23-nucleotide ‘guide sequences’. RNA amplification appears to be able to occur in whole animals. Typically then, the guide RNAs can be incorporated into a protein RNA complex which is cable of degrading RNA, the nuclease complex, which has been called the RNA-induced silencing complex (RISC). This RISC complex acts in the second effector step to destroy mRNAs that are recognized by the guide RNAs through base-pairing interactions. RNAi involves the introduction by any means of double stranded RNA into the cell which triggers events that cause the degradation of a target RNA. RNAi is a form of post-transcriptional gene silencing. Disclosed are RNA hairpins that can act in RNAi.

RNAi has been shown to work in a number of cells, including mammalian cells. For work in mammalian cells it is preferred that the RNA molecules which will be used as targeting sequences within the RISC complex are shorter. For example, less than or equal to 50 or 40 or 30 or 29, 28, 27, 26, 25, 24, 23, 22, 21, 20, 19, 18, 17, 16, 15, 14, 13, 12, 11, or 10 nucleotides in length. These RNA molecules can also have overhangs on the 3′ or 5′ ends relative to the target RNA which is to be cleaved. These overhangs can be at least or less than or equal to 1, 2, 3, 4, 5, 6, 7, 8, 9, 10, 15, or 20 nucleotides long. RNAi works in mammalian stem cells, such as mouse ES cells.

(2) Encode Proteins

The transgenes can also encode proteins or polypeptides. These proteins can be proteins which have a therapeutic value as well as proteins that act to identify the presence of the nucleic acid. Any protein can be expressed from the disclosed nucleic acids for any reason.

f) Features

Disclosed are nucleic acids comprising a promoter and one member of recombinase set, wherein the promoter and the recombinase site are oriented so that when the nucleic acid is attached to a second nucleic acid comprising a transcription start site and a second member of the recombinase set transcription from the transcription start site is increased when a recombinase acts on the recombinase sets.

Also disclosed are nucleic acids comprising a promoter, a set of recombinase sites, and a transcription start site.

Disclosed are nucleic acids, wherein the promoter, the set of recombinase sites, and the transcription start site are oriented so that transcription from the transcription start site is increased when a recombinase acts on the set of recombinase sites.

In one embodiment, LoxP sites can be inserted either between the TATA box and the start site of transcription or flanking the TATA box, with the spacer of the LoxP site centered over the TATA box.

A construct can also have, following the start site of transcription start site, one or more, such as two, copies of the termination sequence for RNA polymerase III dependent transcripts, a poly T tract, can be placed to prevent transcription from the promoter prior to recombination.

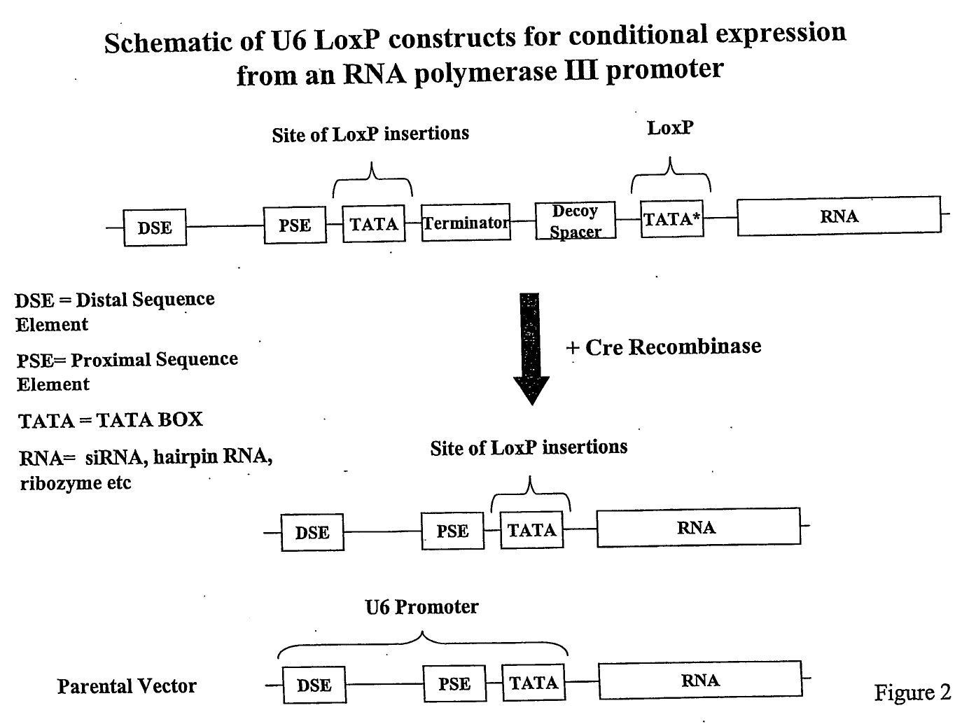

Restriction sites can be placed in the disclosed nucleic acids to facilitate engineering of the molecules. For example, in FIG. 2 there is a depiction of a BpmI site just following the start site of transcription. In FIG. 2 is also depicted, following the terminator sequences, a SmaI restriction site for insertion of a decoy spacer. This decoy spacer can be used to space out the LoxP sites for optimal recombination. Additionally, this site can be used to insert sequences which can be used as a reporter for plasmid delivery, transgenesis, and/or recombination efficiency. FIG. 2 also depicts, following the spacer site, a BglII restriction site. This site can be used in conjunction with the BpmI site to clone in sequences using the same overhangs as those used to clone into the downstream cloning site with BseRI and BamHI. This site is then followed by a LoxP site that is identical to and in the same orientation as the LoxP sequence used above in the vicinity of the TATA box. In some cases, it can contain a TATA box.

FIG. 2 also shows an embodiment in which the second LoxP site is anteceded by a second cloning site. This site is the site that should be used to clone in the sequence whose expression is to be regulated, i.e. the transgene. In these versions, BseRI and BamHI are used as the cloning sites. However, the restriction enzymes used here and above can be substituted for as needed.

Also disclosed are cells and animals comprising the disclosed nucleic acids.

g) RNA Polymerases

There are typically considered three types of eukaryotic polymerases, RNA pol I, II, and III and one type of prokaryotic RNA polymerase. There are a number of differences between these types of polymerase, one of which is the type of promoter region they activate transcription from. A promoter can be considered a region of DNA to which RNA polymerase binds before initiating the transcription of DNA into RNA. The nucleotide at which transcription starts is designated +1 and nucleotides are numbered from this with negative numbers indicating upstream nucleotides and positive downstream nucleotides. RNA pol I recognizes a single promoter for the precursor of rRNA. RNA polymerase II is the polymerase involved in making mRNA, RNA that codes for proteins and polypeptides. Most RNA pol II have the Goldberg-Hogness or TATA box that is centered around position −25 and has the consensus sequence 5′-TATAAAA-3′. Several promoters have a CAAT box around −90 with the consensus sequence 5′-GGCCAATCT-3′. Typically promoters for genes for “housekeeping” proteins contain multiple copies of a GC-rich element that includes the sequence 5′-GGGCGG-3′. Transcription by polymerase II can also be affected by more distant elements known as enhancers.

RNA polymerase III synthesizes 5s ribosomal RNA, all tRNAs, and a number of small RNAs. The promoter for RNA polymerase III is located within the gene either as a single sequence, as in the 5s RNA gene, or as two blocks, as in all tRNA genes. RNA polymerase III transcription initiation requires a number of factors. Three factors are transcription factor IIIB (TFIIIB), which directly contacts RNA polymerase III and is sufficient to support several rounds of RNA polymerase III transcription in yeast, once at the promoter, TFIIIB (Paule, M. and White, R. 2000. Survey and summary: Transcription by RNA polymerases I and III. Nucleic Acids Res. 28: 1283-1298, which is herein incorporated by reference at least for material related to pol III transcription). Yeast TFIIIB consists of three subunits. The first subunit is the TATA box-binding protein TBP (Kassavetis et al. 1992, “The role of the TATA-binding protein in the assembly and function of the multisubunit yeast RNA polymerase III transcription factor,” TFIIIB. Cell 71: 1055-1064; Kassavetis, G. A., et al., 1997, “Domains of the Brf component of RNA polymerase III transcription factor IIIB (TFIIIB): Functions in assembly of TFMB-DNA complexes and recruitment of RNA polymerase to the promoter.” Mol. Cell. Biol. 17: 5299-5306; Kassavetis, G. A., et al., 1998. A post-recruitment function for the RNA polymerase III transcription-initiation factor IIIB. Proc. Natl. Acad. Sci. 95: 9196-9201, all of which are herein incorporated by reference at least for material related to pol III transcription). The second subunit is the TFIIB-related factor, BRF (TDS4/PCF4) (Buratowski, S. and Zhou, H. 1992, “A suppressor of TBP mutations encodes an RNA polymerase III transcription factor with homology with TFEB,”. Cell 71: 221-230; Colbert, T. and Hahn, S. 1992, “A yeast TFIIB-related factor involved in RNA polymerase III transcription. Genes & Dev. 6: 1940-1949; López-De-León, A., et al., 1992, “PCF4 encodes an RNA polymerase III transcription factor with homology to TFIIB. Cell 71: 211-220 all which are herein incorporated by reference at least for material related to pol III transcription). The third subunit is the B″ protein (TFIIIB90/TFC5/TFC7) (Kassavetis, G. A., et al., 1995. Cloning, expression, and function of TFC5, the gene encoding the B” component of the Saccharomyces cerevisiae RNA polymerase III transcription factor TFIIIB. Proc. Natl. Acad. Sci. 92: 9786-9790; Roberts, S., et al., 1996, “Cloning and functional characterization of the gene encoding the TFIIIB90 subunit of RNA polymerase III transcription factor TFIIIB,” J. Biol. Chem. 271: 14903-14909; Rüth, J., et al., 1996, “A suppressor of mutations in the class III transcription system encodes a component of yeast TFIIIB. EMBO J. 15: 1941-1949 all which are herein incorporated by reference at least for material related to pol III transcription).

Most prokaryotic promoters contain two consensus sequences that are involved in recognition and binding of the polymerase. The first, the Pribnow box, is at about −10 and has the consensus sequence 5′-TATAAT-3′. The second, the −35 sequence, is centered about −35 and has the consensus sequence 5′-TTGACA-3′.

2. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

3. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization can involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions can provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

4. Nucleic Acids

There are a variety of molecules disclosed herein that are nucleic acid based, including for example the nucleic acids that encode, for example polypeptides, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556),

91. A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or 0) at the C6 position of purine nucleotides.

b) Sequences

There are a variety of sequences related to for example, functional nucleic acids or promoters or recombinase sites, these sequences and others are herein incorporated by reference in their entireties as well as for individual subsequences contained therein.

It is understood that the description related to this sequence is applicable to any sequence related to the compositions disclosed herein unless specifically indicated otherwise. Those of skill in the art understand how to resolve sequence discrepancies and differences and to adjust the compositions and methods relating to a particular sequence to other related sequences. Primers and/or probes can be designed for any sequence given the information disclosed herein and known in the art.

c) Primers and Probes

Disclosed are compositions including primers and probes, which are capable of interacting with, for example, the functional nucleic acids, as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner.

5. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991) Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as the promoter-recombinase nucleic acids into the cell without degradation and include a promoter yielding expression of the gene in the cells into which it is delivered. In some embodiments the vectors are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for hiterleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

(3) Adeno-Asscociated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and can contain upstream elements and response elements.

(4) Large Payload Viral Vectors

Molecular genetic experiments with large human herpes viruses have provided a means whereby large heterologous DNA fragments can be cloned, propagated and established in cells permissive for infection with herpes viruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials can be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447451, (1991); Bagshawe, K. D., Br. J. Cancer, 60:275-281, (1989); Bagshawe, et al., Br. J. Cancer 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol. 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research. 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

c) In Vivo/Ex Vivo

As described above, the compositions can be administered in a pharmaceutically acceptable carrier and can be delivered to the subject=s cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

6. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and can contain upstream elements and response elements.

a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer can be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker can be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

As discussed herein there are numerous variants of proteins, such as marker proteins, that are known and herein contemplated. Protein variants and derivatives are well understood to those of skill in the art and in can involve amino acid sequence modifications. For example, amino acid sequence modifications typically fall into one or more of three classes: substitutional, insertional or deletional variants. Conservative modifications are understood and would include, for example substitutions which do not differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein in the particular organism from which that protein arises is also known and herein disclosed and described.

7. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material can be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions can be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials can be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer 58:700-703, (1988); Senter, et al., Bioconjugate Chem. 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta. 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers can be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Compositions for oral administration include powders or granules, suspensions or solutions in water or non-aqueous media, capsules, sachets, or tablets. Thickeners, flavorings, diluents, emulsifiers, dispersing aids or binders can be desirable.

Some of the compositions can potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions can be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

8. Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

9. Computer Readable Mediums

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

Disclosed are computer readable mediums comprising the sequences and information regarding the sequences set forth herein.

10. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended. For example, disclosed are kits containing any of the disclosed nucleic acids and/or reagents for their manipulation.

### D. METHODS OF MAKING THE COMPOSITIONS

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed proteins, such as SEQ ID NO:23, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides can be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

3. Process Claims for Making the Compositions

Disclosed are processes for making the compositions as well as making the intermediates leading to the compositions. There are a variety of methods that can be used for making the compositions, such as synthetic chemical methods and standard molecular biology methods. It is understood that the methods of making these and the other disclosed compositions are specifically disclosed.

Disclosed are nucleic acid molecules produced by the process comprising linking in an operative way the disclosed nucleic acids and a sequence controlling the expression of the nucleic acid.

Disclosed are cells produced by the process of transforming the cell with any of the disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are non-human organisms, such as animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

Also disclose are animals produced by the process of adding to the animal any of the cells disclosed herein. Also disclosed are vector comprising the disclosed nucleic acids.

Also disclosed are cells, vectors, non-human organisms, non-human animals, non-human mammals, etc that comprise the disclosed nucleic acids.

### E. METHODS OF USING THE COMPOSITIONS

1. Methods of Using the Compositions as Research Tools

The disclosed compositions can be used in a variety of ways as research tools. For example, the disclosed compositions, can be used to transfer nucleic acid material to cells or animals so that the material is expressed in an inducible or tissue specific way.

The compositions can be used for example as targets in combinatorial chemistry protocols or other screening protocols to isolate molecules that possess desired functional properties, such comprising recombinase sites which still act as recombinase sites, but which have improved inhibition activity of the promoter or improved activity of the promoter after the recombination site has undergone recombination.

The disclosed compositions can be used as discussed herein as either reagents in micro arrays or as reagents to probe or analyze existing microarrays. The disclosed compositions can be used in any known method for isolating or identifying single nucleotide polymorphisms. The compositions can also be used in any method for determining allelic analysis. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

### F. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Cre Mediated RNA Knockdown

Until recently, there were only three principle ways to abolish expression of a gene: via antisense, ribozyme, or knockout. Knockouts are extremely time consuming and challenging. Antisense technology has drawbacks because regulated expression of the antisense molecules has not yet been achieved. Recently, however, a technique known as RNA interference (RNAi) has been extended to mammalian cells to knockdown expression of target genes.

First identified by Andrew Fire et. al., RNAi is a natural phenomenon that involves double stranded RNA-mediated degradation of target mRNA [{38}]. Recent studies of the mechanisms underlying RNAi have revealed that the success of this technique of gene knockdown is due to the use of an endogenous mRNA silencing system that involves a number of proteins, collectively referred to as the RNA-induced silencing complex (RISC). Originally, application of this technique was limited to plants and lower eukaryotes, such as C. elegans, a nematode which has had nearly all of its genes inactivated experimentally by RNAi [{39}]. Attempts to extend the utility of this method to mammalian cells were unsuccessful due to activation of the double-stranded RNA activated protein kinase, PKR, and subsequent shut down of cellular functions [{39}]. However, within the past year, several groups have successfully circumvented PKR activation by using short interfering dsRNA (siRNA), that do not trigger a PKR response [{40}]. Since these original applications in mammalian cells, the interest and use of RNAi to effect transient gene knockdown has exploded and been used to silence expression of a wide array of genes in vitro, from the abundant lamin A/C to the p53 tumor suppressor [{40}]. A further improvement on the utility of RNAi was the development of DNA based vectors to express short hairpin dsRNA's that mimic endogenous micro-RNA's and effect RNAi, allowing for stable suppression of gene expression [{40}].

Study of gene function in vivo in higher eukaryotes (e.g. mice) has historically been conducted through targeted deletion, or knockout, of the gene of interest. Targeted knockout of a gene has traditionally been achieved via replacement of coding sequence with another sequence, such as a β-galactosidase reporter cassette, using homologous recombination. Achieving such a replacement is a time consuming and difficult endeavor, to say the least Due to the difficult cloning often involved with construction of the targeting construct and the large number of cells that need to be screened for homologous recombination, the time invested in generation of a knockout in this fashion can take years. Additionally, this technique is limited due to complete loss of gene expression which often results in embryonic lethality, precluding any studies of function in adult animals and experimental disease models. This latter limitation can be circumvented by using recombinase-mediated excision.

Gene knockout using recombinase-mediated excision is based on the ability of the recombinase to recognize specific, non-native sequences, or recognition sites, and excise intervening sequence between two similarly oriented recognition sites [{53}]. Here homologous recombination is used to introduce recombinase recognition sites, such as the LoxP site, flanking the gene to be knocked out, resulting in a “floxed” locus. When homozygous floxed mice are crossed with a mouse line expressing a recombinase, such as Cre recombinase, the DNA between the LoxP sites is excised, effectively removing the gene of interest [{53}]. By expressing Cre recombinase from a tissue specific or inducible promoter, site-directed excision can be controlled both spatially and temporally [{54}]. Additionally, Cre fusion proteins have been generated whose activity can be regulated in a ligand-dependent manner, adding an additional level of control [{55}]. Despite the exquisite control afforded with these systems, they are similarly limited as conventional knockouts due to their reliance on the homologous recombination method.

The development of a more rapid and less technically challenging method for suppressing gene expression could significantly alter the way in which gene function is studied in vivo and would accelerate the whole discovery process in general. Disclosed is a system with high-throughput screening of gene function in vivo. Disclosed are systems which utilize cloning of a targeting construct, generation of heterozygotes for study, and which is capable of undergoing both constitutive and conditional expression. With this last condition, the disclosed systems utilize conditional expression systems, such as Cre recombinase.

RNAi harnesses a natural regulatory system to regulate gene expression. Originally achieved by delivering ex vivo-generated short dsRNA (siRNA) to cells, an alternate method has been recently developed that makes use of DNA-based vectors to generate a dsRNA hairpin that can function in the same capacity of siRNA [{40}]. These vectors, as mentioned above, can be used to stably suppress gene expression. They can also be used in theory to generate transgenic knockdown animals. Although RNAi has been used to a limited extent to generate transgenic knockdown in other organisms, such as Drosophila [{57}]. RNAi can be effected in adult mice, constitutive expression is not likely preferred [{39}]. Reports indicate that transient knockdowns have similar phenotypes to their knockout counterparts [{58}].

The DNA-based RNAi technique is limited by the use of a RNA polymerase III promoter. These promoters are used for their high degree of transcript production and precise termination of transcription, in contrast to RNA polymerase II promoters that leave long poly A tails on their transcripts. Due to their constitutive nature, RNA polymerase III promoters, such as the U6 promoter, would be limited in their application to transgenics to global knockdown. Generation of a conditional RNA polymerase III promoter would thus be precursor to this technology becoming a viable alternative to targeted knockouts. While some inducible U6 promoters have been described, these promoters are limited either due to their requirement of an obscure regulation system or due to their being in an on state in the absence of a regulator. Mice created with this latter condition would have to go through at least one generation in which the hairpin would be expressed.

Disclosed is a system for conditional expression from, a promoter, such as an RNA Polymerase III, such as a U6 promoter, or an RNA polymerase II promoter. The system comprises a conditional expression system, involving a recombinase, such as the Cre-recombinase system. The disclosed systems allow for targeted expression of hairpins from, for example a U6 promoter, in transgenic mice.

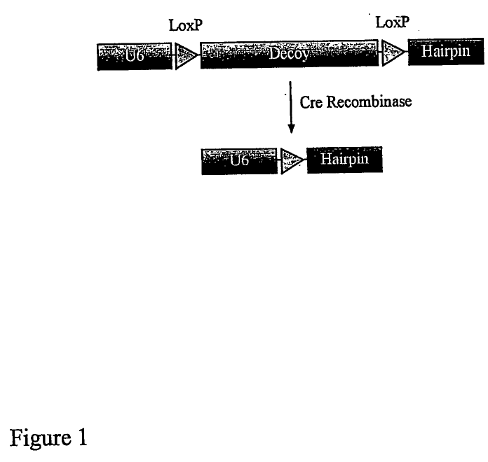

This system can comprise an insertion of a decoy spacer that separates the RNA Pol III or RNA Pol II or RNA Pol I promoter, such as a U6 pol III promoter and the functional transgene, such as an RNA expression cassette, such as a RNA hairpin cassette. In this configuration the expression of the hairpin is prevented (FIG. 1). In the presence of Cre, the intervening decoy spacer is excised and the RNA Pol III promoter, such as the U6 promoter, or RNA Pol II promoter is brought into proximity of the functional RNA cassette hairpin cassette, allowing for expression.

The disclosed systems work, even if the promoter has a low tolerance for mutation, such as the U6 promoter. Four different U6-loxP constructs that either incorporate wild type or mutant loxP sites in front of or superimposed on the TATA box were generated. While all four promoters were functional, those superimposed over the TATA box were unaffected by insertion of the LoxP sites. Furthermore, all loxP mutants were equal to or better than a wild-type loxP site in recombination efficiency. Thus these U6-promoter constructs can be used to effect conditional, targeted expression of hairpins in conjunction with Cre recombinase.

a) Results

(1) Recombinase Sites Do Not Alter Promoter Function of RNA pol III Promoters

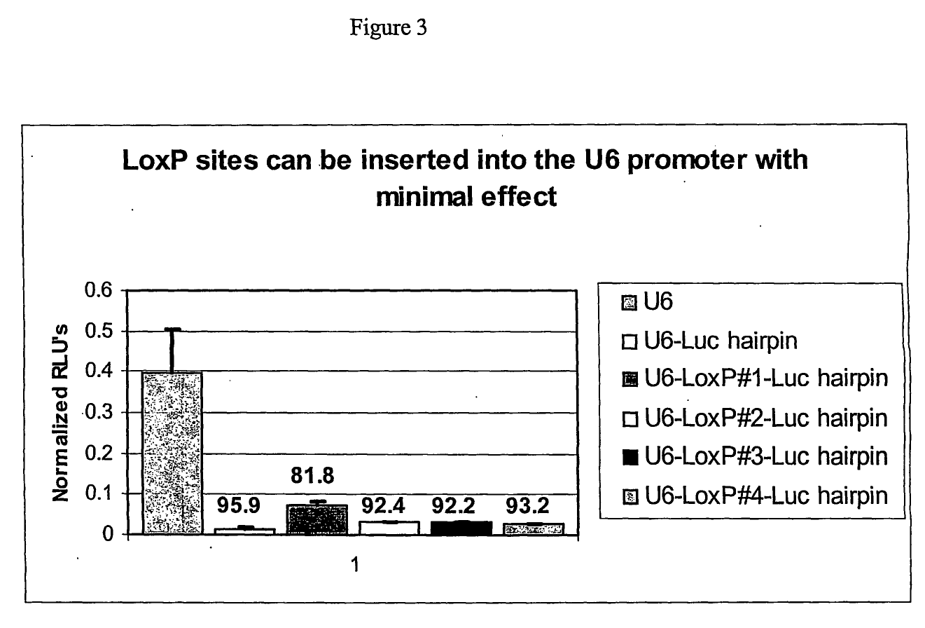

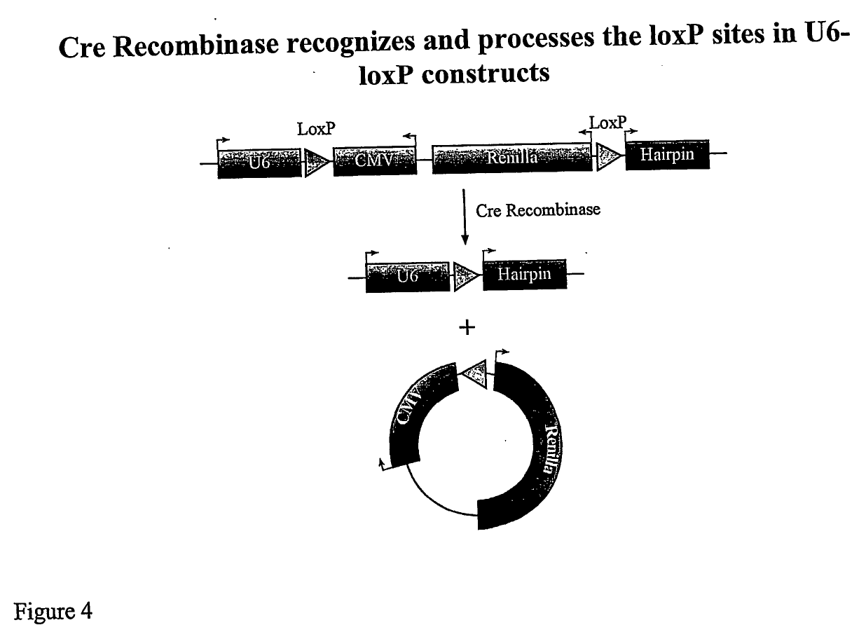

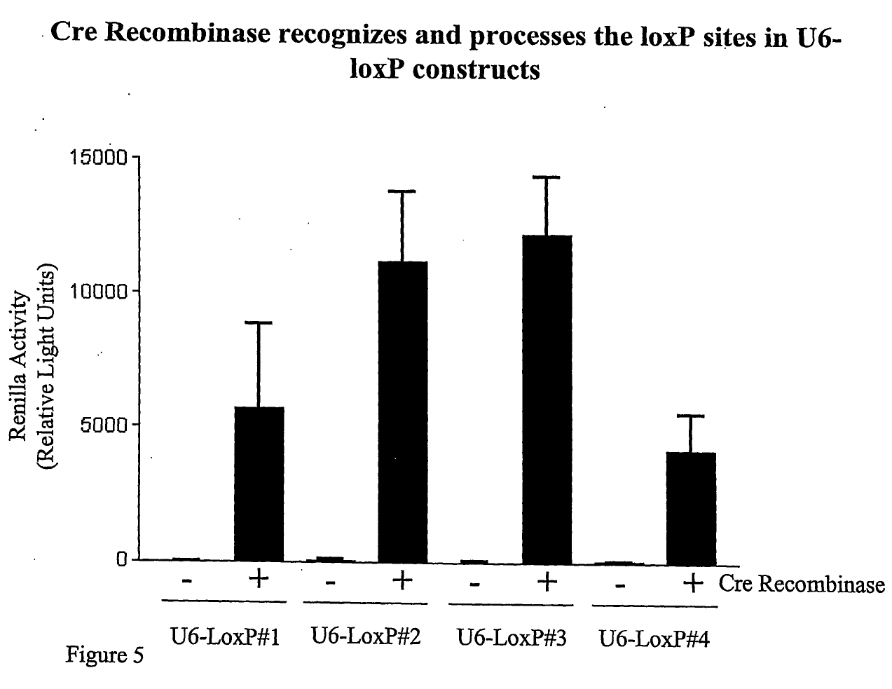

To test if insertion of a LoxP site alters U6 promoter function, a hairpin (Luc) targeted against the firefly luciferase gene was cloned downstream of the promoter in each U6-LoxPconstruct U6-loxP-Luc constructs were then tested for their ability to silence expression of transiently overexpressed luciferase. In all cases, the U6-loxP constructs were functional and only construct #1, which has the loxp site inserted downstream of the TATA box, showed reduced activity as compared to the parental promoter (FIG. 3). This indicates that U6-LoxP constructs are functional so that following recombination, a RNA can be expressed from these constructs.

(2) Recombinase Sites Do Not Alter Promoter Function of RNA Pol II Promoters

Cre-recombinase mediated recombination of U6-loxP constructs was tested as follows: a renilla luciferase gene and a CMV promoter were inserted (@SmaI site) between the loxP sites in the U6-loxP constructs. The renilla gene and CMV promoter were orientated such that the CMV promoter and the renilla gene are separated prior to recombination. Note: this approach is based on the same principle employed for the regulation of RNA Pol III promoters with Cre recombinase. In the absence of Cre, the renilla gene is not expressed. In the presence of Cre, recombination occurs and the CMV promoter is now adjacent to the renilla gene, allowing for expression and detection of renilla luciferase. Two of the U6 constructs (#2 and #3) showed enhanced recombination efficiency as compared to the construct containing a wild type loxP site (#1).

### 2. Example 2

### AKAP12β

Knockdown of AKAP12β can be performed by generating transgenic mice expressing a dsRNA hairpin targeted against the AKAP12β mRNA. Two lines of mice will be generated: the first line will be a constitutive line in which the hairpin is expressed from a native U6 promoter, resulting in global expression; the second line made has expression of the hairpin restricted to vascular SMC using a Cre recombinase-regulated U6 promoter which has been generated. The efficacy of this system can be concurrently tested with an EGFP hairpin reporter line. Expression of hairpins and AKAP12β knockdown can be confirmed by RNAse protection assay. These lines can then be used in a ligation injury model to examine the effect of AKAP12β on neointimal formation. The results indicated that the U6-loxP promoters and RNAi against AKAP12β in vitro, can suppress AKAP12β expression in transgenic mice and 2-AKAP12β knockdown mice will show accelerated lesion formation following vascular injury.

To knockdown AKAP12β expression in SMC in vivo, transgenic mice can be generated that carry a constitutively expressed AKAP12β hairpin or the U6-decoy-AKAP12β hairpin described above. Similar mice can be generated that express a hairpin targeted against EGFP to control for any effects of hairpin expression. These mice can also be used to generate an indicator line via cross with CMV-EGFP mice (Jackson Labs) to assess the functionality of RNAi in transgenic mice. In order to facilitate detection of positive mice, the decoy used can be either LacZ or a red fluorescent protein (Red2-nuc) driven be the CMV promoter. Positive mice (n≧2) will then be crossed with either SM22-Cre or SM22-CreERT2 mice, which are possessed, to remove the decoy spacer (CMV-LacZ or CMV-Red2-nuc) and bring the hairpin under control of the U6 promoter only in SM22 expressing tissues (e.g. arterial SMC).

Recombination can be assessed by the absence of LacZ or Red2-nuc expression in SMC, while expression of the AKAP12β hairpin and AKAP12β mRNA in the aorta and other SMC-rich tissue can be examined by RPA.

Following confirmation of AKAP12β knockdown and characterization of normal vessel histology in these animals, ligation of the external carotid artery can be performed to mimic vascular injury. The effect of AKAP12β on neointimal formation can then be examined in knockdown mice versus the parental lines that have not undergone recombination. SMC proliferation can be assessed by BrdU incorporation and vascular remodeling and neointimal formation can be quantitated by morphometry. These knockdown mice are expected to enhance SMC proliferation following injury.

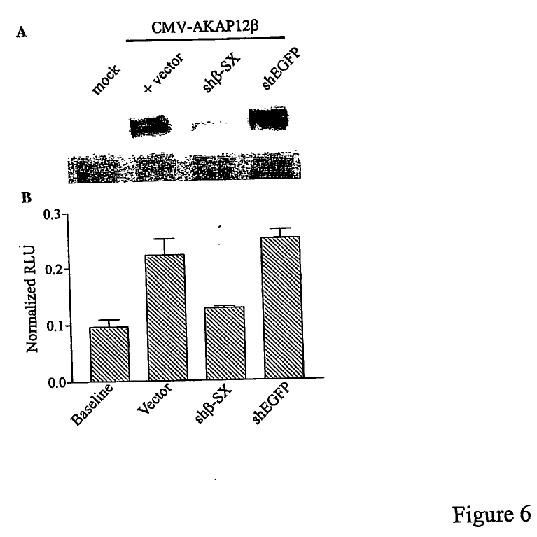

Using a hairpin designed against the luciferase gene, it was demonstrated that RNAi is functional in both primary and clonal SMC cell lines. A hairpin was designed to AKAP12β. The reliance of RNAi on short sequences allows for targeting of specific exons of a gene, making this technique ideal for the study of splice variants and other isoforms of a gene. As AKAP12β differs from the other isoforms by only one exon (exon 3), the potential target sequence was limited to the 5′ UTR of AKAP12β. Following creation and testing of three potential hairpin vectors, one such hairpin vector that effectively and specifically suppresses expression of AKAP12β (FIG. 6) has been generated.

The ability to knockdown AKAP12β expression in vivo and in vitro allows for thorough analysis of the role of AKAP12β in regulating SMC proliferation and PKA activity. As AKAP12 is the only retinoid-responsive tumor suppressor identified to date, the preliminary data presented in this aim coupled with Dr Gelman's observations that SSeCKS is a modulator of growth suggest that AKAP12β can mediate retinoid-induced cell cycle arrest and regulate normal SMC proliferation. Consistent with this, my attempts to produce an AKAP12β overexpressing SMC line were unsuccessful, resulting in either no expression or expression of truncated forms of AKAP12β. Similarly, attempts to generate a CMV-driven AKAP12β adenovirus were unsuccessful due to the lack of plaque formation in HEK293 cells, presumably from a complete growth arrest of these cells from AKAP12β overexpression.

To abolish the in vitro expression of AKAP12β, stable PAC1 lines can be generated that express a double-strand RNA hairpin targeted against AKAP12β. Expression of the hairpin in these lines can be confirmed by RNAse protection assay (RPA) using an in vitro transcribed radiolabeled hairpin as the probe. Knockdown of AKAP12β basal, serum-, and retinoid-stimulated protein expression in positive lines can then be examined by Western blotting. The three cell lines showing the highest levels of hairpin expression and lowest levels of AKAP12β can be used for further analysis. As a control, three cell lines harboring an empty hairpin expression vector will be generated.

The effect of AKAP12β knockdown on basal, forskolin-, and atRA-stimulated changes in PKA activity, SMC growth, cell cycle progression, and apoptosis, can be examined using these cell lines.

a) Methods

There are a variety of methods that can be used to address issues with the AKAPβ RNAi successes. It is understood that these methods can be applied to other systems as well.

(1) SMC Culture and Generation of Stable Cell Lines

PAC1 SMC can be cultured in DMEM containing 10% FBS and no antibiotics (Note: although the PAC1 SMC line can be used throughout this application, cross-validation can be performed where possible using primary rat aortic or human coronary artery SMC to account for cell line artifacts). Quiescence can be achieved by culturing sub-confluent SMC in 0.25% FBS for 24 hours. Transfections can be carried out using either the calcium phosphate method or FuGene6 (Roche) using manufacturer's recommendations. AKAP12β overexpressing PAC stable lines (PAC-tet-AKAP12β can be generated in two steps. First, stable PAC1 lines can be generated by transfecting cells with and selecting for a tet-inducible tet_{R}/VP 16 transactivator (pTet-t,TA). These PAC1-pTet-tTA stable cells can then be co-transfected with either the tet-responsive pUHD10-3-AKAP12β or pUHD10-3-AKAP12Δ construct and a resistance plasmid, such as pTk-Hygro. Following antibiotic selection, individual resistant clones can be isolated, amplified and tested by Western blotting for tet-regulated AKAP12β expression. These cell lines can be cultured with 0.5 μg/ml of tetracycline (tet) (Sigma). To induce SSeCKS expression, tet-containing media can be removed, the plates can be washed with PBS, and fresh tet-free media can be added. AKAP12β knockdown cells can be generated by stably transfecting a plasmid containing a hairpin targeted against the 5′ UTR of the AKAP12β mRNA under control of the U6 promoter.

(2) Total RNA Isolation, Northern Blotting, and RNAse Protection Assay

Total RNA can be isolated from duplicate 100-mln plates by following the guanidinium isothiocyanate (GIT)-acid phenol method [{59}]. For Northern Blotting, approximately 20 μg of total RNA can be fractionated through a 1.2% agarose gel containing 0.66 mol/L formaldehyde. The gel can be washed in 10×SSC and blotted to a nylon membrane. After UV crosslinking the RNA, the blot can be prehybridized at 65° C. with RapidHYBE (Amersham) for one hour. A random primed radioactive labeled cDNA probe can be applied to the prehybridization solution, and incubation can be continued for one hour. Blots can be washed at room temperature twice with 2×SSC/1% SDS for 20 minutes each, followed by at most one 20 minute wash at 55° C. with 1×SSC/0.5% SDS. Blots can be wrapped in plastic wrap and exposed to x-ray film at −80° C. Membranes can be stripped and reprobed with GAPDH or β-actin to serve as a control. RNAse protection assays can be performed using the HybSpeed RPA kit (Ambion) per manufacturers directions.

(3) Total Protein Isolation and Western Blotting

For Western blotting, cells can be lysed in “crack buffer”(50 mM Tris-HCl (pH, 6.8), 100 mM DTT, 100 μg/ml PMSF, 2% SDS, 10% glycerol, 1 μg/ml each of pepstatin A, leupeptin, and aprotinin, and 1 μm sodium orthovanadate), and sheared with a 22 gauge needle. The protein content of the samples can be estimated using the DC protein assay (BioRad). Protein (10-20 μg) can be resolved using sodium dodecyl sulfate-polyacrylamide gel electrophoresis (SDS-PAGE) with 10% SDS and blotted on reinforced nitrocellulose membranes (Amersham) or PVDF (Millipore). Membranes can then be blocked with 5% milk one hour and then be incubated with the appropriate primary antibody for one hour to overnight. After three TBS-T washes, an appropriate secondary antibody linked to horseradish peroxidase can be applied and the membrane incubated for one hour. Following incubation with appropriate secondary antisera, immunoreactive products are detected with a chemiluminescent kit (Pierce). Primary antibodies used include AKAP12 (1:5000; kindly provided by Dr. Irwin Gelnan, Mt. Sinai), basic Calponin (1:5000; Sigma), SM α-actin (1:5000; Sigma), CREB (1:1000; Cell Signaling), phospho-CREB Ser133 (1:200; Cell Signaling), VASP (1:5000; Calbiochem), FLAG (1:5000, Sigma), and EGFP (1:2000; Clontech).

(4) FACS Analysis

Parental PAC, control, and AKAP12β overexpressing/knockdown lines can be grown to 40% confluence, quiesced in 0.25% FBS for 48 hours, washed, and then stimulated with 10% serum +/−2 μm atRA of 1 μM forskolin for 6, 12, 24, 36, 46, and 72 hours. At the indicated time point, cells can be harvested by trypsinization, washed with PBS, and then fixed in 70% ethanol overnight. Cells can be washed and DNA stained with propidium iodide (50 μg/ml) in the presence of 20 μg/ml RNAse for 30 minutes at room temperature. Following washing, cell samples can be analyzed on a FACSCalibur flow cytometer (BD Biosciences). Data can be visualized and analyzed using the CellQuest software package (BD Biosciences).

(5) Comparative Genomics

Genomic sequences encompassing the mouse, rat, and human AKAP12 loci can be obtained from NCBI (http://www.ncbi.nlm.nih.gov/) or Celera (subscription). Comparative analysis of these loci can be performed to identify conserved (>70% identity over 20 nucleotides) non-coding regions using either the PIP (http://nog.cse.psu.edu/pipmaker/) or VISTA (http://www-gsd.lbl.gov/vista/) algorithms. Conserved non-coding regions can be scanned for potential transcription factor binding sites using TRANSFAC (http://transfac.gbf de/TRANSFAC/) or rVISTA (http://nemo.lbl.gov/rvista/index.html).

(6) PCR Cloning, Site Directed Mutagenesis, and Oligo Cloning

All AKAP12 promoter constructs can be cloned initially from total rat genomic DNA or a rat BAC clone. Deletions can be made by PCR as described [{60}] or by restriction digestion. Site-directed mutagenesis of putative RAREs, other potentially relevant cis elements and the AKAP domain of AKAP12β can be carried out with a commercial kit (QuikChange, Stratagene) as described [{61}]. Oligo cloning can be conducted by cutting out short DNA segments flanked by unique restriction sites and then replacing the intervening DNA with the annealed oligos containing the desired sequence (Integrated DNA Technologies). All constructs can be sequenced for conformation (Nucleic Acids Service, Functional Genomics Core).

(7) Luciferase Reporter Assays

The original calcium phosphate co-precipitation method of transfecting DNA can be used to introduce promoter constructs into PAC1 SMC while FuGENE 6 (Roche) can be used to transfect the PathDetect CREB trans-reporting assay system (Stratagene). Briefly, PAC1 SMC can be seeded in 24-well dishes (20,000 cells/well) and transfected at 50-75% confluency. Reporter genes (0.5-1 μg/well) and a control plasmid (50 ng/well of tk-Renilla, Promega) can be added to quadruplicate samples and assayed 48 hours later with a commercial kit (Dual Luciferase Assay, Promega) using a Berthold Luminometer. Luciferase activity can be normalized to tk-Renilla and for promoter assays, expressed as a fold-change from the pGL3 basic vector (promoter-less). Data can be analyzed by ANOVA and Tukey's post-hoc analysis. Significance can be assumed if p<0.05.

(8) Growth and Cell Viability Assays

The effect of AKAP12β over/under-expression on SMC growth can be assessed in the following manner. PAC1 SMC can be plated out on 24 well dishes in triplicate per condition at initial density of 5000 cells/mL and allowed to attach overnight. Cells can then be quiesced for 24 hours in 0.25% FBS. One set of wells can then be trypsinized and counted with a hemocytometer. This number can represent time 0. Daily cell counts can then be performed up to 5 days post-treatment. Conditions to be examined include serum +/−tetracycline for AKAP12β overexpression and serum +/−2 μM atRA or 1 μM forskolin for AKAP12β knockdown studies. Data can be analyzed by a one-way ANOVA using GraphPad Prism Software (San Diego, Calif.). Cell Viability assays can be performed using the CellTiter-Glo luminescent cell viability assay (Promega) as per manufacturers directions.

(9) Apoptosis Assays

Subconfluent PAC1 cells or AKAP12β overexpessing/knockdown lines can be treated with 2 μM atRA or 1 μM forskolin for 24 hours and then fixed with 2% buffered paraformaldehyde and stained with DAPI (1 μg/ml; Molecular Probes). An Olympus IX70 fluorescence microscope can be used then to assess apoptosis as DAPI staining is augmented in apoptotic cells. Ten random fields of cells can be counted and used to calculate an apoptotic index (percent positive staining). This index can then be used for comparison with other treatments. TUNEL assays can be performed with a commercial kit from Roche Biochemicals. Cell fixing and assays can be performed per manufacturer's recommendations.

(10) Immunofluorescence Microscopy

Parental PAC1 or AKAP12 overexpressing or knockdown cells can be grown on chamber slides to 50-60% subconfluence. Media can be rapidly removed and cells can be immediately fixed with room temperature (R1) 4% paraformaldehyde for 10 minutes at RT. Following fixation, cells can be washed twice with PBS and then permeabilized with either ice cold 100% acetone at −20° C. or with 0.1% Triton X-100 at RT for 5 minutes. Cells can then be washed twice with TBS-T and incubated with primary antisera diluted in TBS-T for 1 hour. Primary antisera can then be removed, cells washed twice with TBS-T, and then incubated with secondary antisera conjugated with a fluorophore for 40 minutes. Leaving the secondary antisera on, fluorescently-labeled phalloidin and DNAse I can be added to stain F- and G-actin, respectively, for 20 minutes. Cells can then be washed and in some cases treated with RNAse followed by staining with DAPI to visualize DNA. The chambers of the slide can then be removed, the slide cleaned and coverslipped with AquaMount (LernerLabs). Immunoreactivity and staining can be visualized and recorded on a fluorescence microscope fitted with a digital camera. The F:G actin ratio can be calculated as described [{46}].

(11) Migration Assays

For scrape wound assays, parental PAC, overexpressing, and/or knockdown AKAP12β cell lines can be grown to near confluency on 60 mm plates, then quiesced for 24 hours in 0.25% PBS. A pipette tip can be drawn across the plate in 3 different locations to create 3 reproducible injuries to the cultured cells, then the cells can be washed once with PBS and refed either 0.25%, 10% FBS, or 0.25% FBS containing 20 ng/ml PDGF-BB. At this time, three randomly chosen locations on each wound can be marked. Images of the initial wounds can be captured using a digital camera attached to a microscope. At 12, 24, and 36 hours, the number of cells that migrate into each wound at the marked locations can then be measured by comparison to initial wound images. The mean value of these points can be used to compare migration rates between treatments. Boyden Chamber migration assays can be performed using a 96 well migration assay kit from Chemicon International.

(12) Transgenesis

Mouse pronuclear injections can be carried out as known, or by for example, commercial facilities. F₀ founder transgenics can be identified by fluorescent detection of DsRed2 or by LacZ staining of tissue harvested from tail snippets of potential founders and weaned offspring. Transgenic founders can then be mated to FVB mice for the establishment of transgenic lines.

(13) Ligation Injury

The carotid artery flow cessation, ligation model of neointimal formation [{62}] has been routinely used in our lab and can be used for studies of AKAP12β knockdown on neointimal formation and at RA-mediated effects. The anesthetized mouse is placed with its back onto a platform with rubber bands attached to its hind legs. The rubber bands are attached to the screws sticking out from the side of the platform thereby stretching the legs. Another rubber band is used to hold down the head by the incisors. The front legs can be kept away from the operating field with tape. The ventral part of the neck is shaved and the skin disinfected with iodine (Betadine or Povidone iodine). A midline incision on the ventral side of the neck (1-1.5 cm) is made and the salivary glands moved laterally by blunt dissection. Usually the procedure is performed on the left carotid artery since this vessel has no side branches (right side has subClavian artery) and endothelial re-growth can thus only occur from the carotid bifurcation and the aortic arch. For better access to the left carotid artery, the left salivary gland is held out of the way by the DeBakey clamp. The straight neck muscles are pushed medially while blunt dissection of the external carotid artery is performed with the micro dissecting forceps with curved tips. These forceps are very suitable for separating the connective tissue from the vessel and placing the ligature. The common, internal, external carotid artery or femoral artery is then permanently tied off with a 6-0 silk ligature just proximal of the carotid bifurcation. In some studies, a similar ligation to the femoral artery by exposure through a femoral cut down can be performed. To test whether clotting of the vessel occurred, the common carotid artery can be monitored for pulsation and color of the blood. In addition, if blood flows out of the hole briskly, clotting usually did not occur. Following successful ligation of the carotid/femoral artery, the wound can be closed with a standard surgical staple gun and cleansed of blood with sterile gauze wetted with iodine. The animal can be placed under a warm lamp and closely monitored with respect to surgical plane (as indicated above) and visual breathing until it regains consciousness at which time the animal can be placed into a plastic cage without bedding material. At select time points, animals can be sequentially perfused with PBS (5 minutes) followed by 2% paraformaldehyde (10 minutes) and both left and right carotid or femoral arteries can be extracted and fixed. All subsequent histological work can be carried out in conjunction with the CCVR Histopathology Core Facility (Mary Georger). BrdU labeling and detection as well as morphometry can be performed as described [{63}]

### G. REFERENCES

1. Ross R. Atherosclerosis: A defense mechanism gone awry. Am J Pathol 1993; 143(4):987-1002.
2. Davies M G, Hagen P O. Pathobiology of intimal hyperplasia. Brit J Surg 1994; 81:1254-1269.
3. Sata M, Saiura A, Kunisato A, Tojo A, Okada S, Tokuhisa T, Hirai H, Makuuchi M, Hirata Y, Nagai R. Hematopoietic stem cells differentiate into vascular cells that participate in the pathogenesis of atherosclerosis. Nat Med 2002; 8(4):403-409.
4. Simper D, Stalboerger P G, Panetta C J, Wang S, Caplice N M. Smooth muscle progenitor cells in human blood. Circulation 2002; 106:1199-1204.
5. Hu Y, Davison F, Ludewig B, Erdel E, Mayr M, Url M, Dietrich H, Xu Q. Smooth muscle cells in transplant athersclerotic lesions are originated from recipients, but not bone marrow progenitor cells. Circulation 2002; 106:1834-1839.
6. Evans R M. The steroid and thyroid hormone receptor superfamily. Science 1988; 240:889-895.
Streb J W, Miano J M. Retinoids: pleiotropic agents of therapy for treatment of vascular disease? Current Drug Targets 2003; Submitted.
8. Strickland S, Mahdavi V. The induction of differentiation in teratocarcinoma stem cells by retinoic acid. Cell 1978; 15:393-403.
9. Warrel R P, de The H, Wang Z Y, Degos L. Acute promyelocytic leukemia. New Engl J Med 1993; 329:177-189.
10. Miano J M, Berk B C. Retinoids: Versatile biological response modifiers of vascular smooth muscle phenotype. Circ Res 2000; 87:355-362.
11. Shindo T, Manabe I, Fukushima Y, To be K, Aizawa K, Miyamoto S, Kawai-Kowase K, Moriyama N, Imai Y, Kawakami H, Nishimatsu H, Ishikawa T, Suzuki T, Morita H, Maemura K, Sata M, Hirata Y, Komukai M, Kagechika H, Kadowaki T, Kurabayashi M, Nagai R. Krüppel-like zinc-finger transcription factor KLF5/BTEB2 is a target for angiotensin II signaling and an essential regulator of cardiovascular remodeling. Nat Med 2002; 8(8):856-863.
12. Minucci S, Pelicci P G. Retinoid receptors in health and disease: co-regulators and the chromatin connection. Semin Cell Dev Biol 1993; 10:215-225.
13. Ou H, Haendeler J, Aebly M R, Kelly L A, Cholewa B C, Koiki G, Kwitek-Black A, Jacob H J, Berk B C, Miano J M. Retinoic acid-induced tissue transglutaminase and apoptosis in vascular smooth muscle cells. Circ Res 2000; 87:881-887.
14. Chen J, Maltby K M, Miano J M. A Novel Retinoid-Response Gene Set in Vascular Smooth Muscle Cells. Biochem Bioph Res Co 2001; 281:475-482.
15. Frankfort B J, Gelman I H. Identification of novel cellular genes transcriptionally suppresed by v-src. Biochem Bioph Res Co 1995; 206(3):916-926.
16. Xia W, Unger P, Miller L, Nelson J, Gelman I H. The Src-suppressed C Kinase Substrate, SSeCKS, Is a Potential Metastasis Inhibitor in Prostate Cancer. Cancer Res 2001; 61:5644-5651.
Lin X, Nelson P J, Gelman I H. SSeCKS, a major protein kinase C substrate with tumor suppressor activity, regulates G₁-S progression by controlling expression and cellular compartmentalization of cyclin D. Mol Cell Biol 2000; 20(19):7259-7272.
18. Gelman I H, Lee K, Tombler E, Gordon R, Lin X. Control of cytoskeletal architecture by the src-suppressed C kinase substrate, SSeCKS. Cell Motil Cytoskel 1998; 41:1-17
19. Lin X, Gelman I H. Reexpression of the major protein kinase C substrate, SSeCKS, suppresses v-src-induced morphological transformation and tumorigenesis. Cancer Res 1997; 57:2304-2312.
20. Gordon T, Grove B, Loftus J C, O'Toole T, McMillan R, Ginsberg M H. Molecular cloning and preliminary characterization of a novel cytoplasmic antigen recognized by myasthenia gravis sera. J Clin Invest 1992; 90:992-999.
21. Nauert J B, Klauck T M, Langeber L K, Scott J D. Gravin, an autoantigen recognized by serum from myasthenia gravis patients, is a kinase scaffold protein. Curr Biol 1997; 7(1):52-62.
22. Michel J J, Scott J D. AKAP mediated signal transduction. Annu Rev Pharmacol 2002; 42:235-257.
23. Cassano S, Gallo A, Buccigrossi V, Porcellini A, Cerillo R, Gottesman M E, Avvedimento EV. Membrane localization of cAMP-dependent protein kinase amplifies cAMP signaling to the nucleus in PC12 cells. J Biol Chem 1996; 271(47):29870-29875.
24. Lin X, Tombler E, Nelson P J, Gelman I H. A novel src- and ras-suppressed protein kinase C substrate associated with cytoskeletal architecture. J Biol Chem 1996; 271(45):28430-28438.
Lin X, Gelman I H. Calmodulin and cyclin D anchoring sites on the src-suppressed C kinase substrate, SSeCKS. Biochem Bioph Res Co 2002; 290:1368-1375.
26. Shih M, Lin F, Scott J D, Wang H, Malbon C C. Dynamic complexes of □₂-adrenergic receptors with protein kinases and phosphatases and the role of gravin. J Biol Chem 1999; 274(3):1588-1595.
Xia W, Gelman I H. Mitogen-induced, FAK-dependent tyrosine phosphorylation of the SSeCKS scaffolding protein. Exp Cell Res 2002; 277:139-151.
28. Ferbeyre G. PML a target for translocations in APL is a regulator of cellular senescence. Leukemia 2002; 16(10):1918-1926.
29. Lin X, Nelson P J, Frankfort B, Tombler E, Johnson R, Gelman I H. Isolation and characterization of a novel mitogenic regulatory gene, 322, which is transcriptionally repressed in cells transformed by src and ras. Mol Cell Biol 1995; 15:2754-2762.
30. Nelson P J, Gelman I H. Cell-cycle regulated expression and serine phosphorylation of the myristylated protein kinase C substrate, SSeCKS: correlation with culture confluency, cell cycle phase and serum response. Mol Cell Biochem 1997; 175:233-241.
31. Coats S R, Covington J W, Su M, Pabón-Peña L M, Eren M, Hao Q, Vaughan D E. SSeCKS gene expression in vascular smooth muscle cells: regulation by angiotensin II and a potential role in the regulation of PAI-1 gene expression. J Mol Cell Cardiol 2000; 32:2207-2219.
32. Sato N, Kokama K, Shimokado K, Kato H, Miyata T. Changes of gene expression by lysophosphatidylcholine in vascular endothelial cells: 12 up-regulated distinct genes including 5 cell growth-related, 3 thrombosis-related, and 4 others. J Biochem 1998; 123:1119-1126.
33. Kitamura H, Okita K, Fujikura D, Mori K, Iwanaga T, Saito M. Induction of src-suppressed C kinase substrate (SSeCKS) in vascular endothelial cells by bacterial lipopolysaccharide. J Histochem Cytochem 2002; 50(2):245-255.
34. Thomton J W. Evolution of vertebrate steroid receptors from an ancestral estrogen receptor by ligand exploitation and serial genome expansions. PNAS 2001; 98(10):5671-5676.
35. Bornfeldt K E, Krebs E G. Crosstalk between protein kinase A and growth factor receptor signaling pathways in arterial smooth muscle. Cell Signal 1999; 11(7):465-477.
36. Indolfi C, Stabile E, Coppola C, Gallo A, Perrino C, Allevato G, Cavuto L, Torella D, Di Lorenzo E, Troncone G, Feliciello A, Avvedimento E V, Chiariello M. Membrane-bound protein kinase A inhibits smooth muscle cell proliferation in vitro and in vivo by amplifying cAMP-protein kinase A signals. Circ Res 2001; 88:319-324.
37. Klemm D J, Watson P A, Frid M G, Dempsey E C, Schaack J, Colton L A, Nesterova A, Stenmark K R, Reusch J E B. cAMP response element-binding protein content is a molecular determinant of smooth muscle cell proliferation and migration. J Biol Chem 2001; 276 (49):46132-46141.
38. Fire A, Xu S, Montgomery M K, Kostas S A, Driver S E, Mello C C. Potent and specific genetic interference by double-stranded RNA in Caenorhabditis elegans. Nature 1998; 391:806-811.
39. Hannon G J. RNA interference. Nature 2002; 418:244-251.
40. McManus M T, Sharp P A. Gene silencing in mammals by small interfering RNAs. Nat Rev Genet 2002; 3:737-747.
41. Ohkawa J, Taira K. Control of the functional activity of an antisense RNA by a tetracycline-responsive derivative of the human U6 snRNA promoter. Hum Gene Ther 2000; 11(4):577-585.
42. Miano J M. Mammalian smooth muscle differentiation: origins, markers, and transcriptional control. Results and Problems in Cell Differentiation 2002; 38:39-59.
43. Clowes A W, Schwartz S M. Significance of quiescent smooth muscle migration in the injured rat carotid artery. Circ Res 1985; 56:139-145.
44. Ridley A J. Rho GTPases and cell migration. J Cell Sci 2001; 114:2713-2722.
45. Sandau K B, Gantner F, Brüne B. Nitric oxide-induced F-actin disassembly is mediated via cGMP, cAMP, and protein kinase A activation in rat mesangial cells. Exp Cell Res 2001; 271:329-336.
46. Hirshman C A, Zhu D, Panettieri R A, Emala C W. Actin depolymerization via the beta-adrenoceptor in airway smooth muscle cells: a novel PKA-independent pathway. American Journal of Physiology Cell Physiology 2001; 281(5):C1468-C1476.
47. Cramer L P. ENA/Vasp: Solving a cell motility paradox. Curr Biol 2002; 12(12):R417-R419.
48. Walders-Harbeck B, Khaitlina S Y, Hinssen H, Jockusch B M, Menberger S. The vasodilator-stimulated phosphoprotein promotes actin polymerisation through direct binding to monomeric actin. FEBS Lett 2002; 529:275-280.
49. Bear J E, Svitkina T M, Krause M, Schafer D A, Loureiro J J, Strasser G A, Maly I V, Chaga O Y, Cooper J A, Borisy G G, Gertler F B. Antagonism between Ena/VASP proteins and actin filament capping regulates fibroblast motility. Cell 2002; 109(4):509-521.
50. Halbrugge M, Friedrich C, Eigenthaler M, Schanzenbacher P, Walter U. Stoichiometric and reversible phosphorylation of a 46-kDA protein in human platelets in response to cGMP- and cAMP-elevating vasodilators. J Biol Chem 1990; 265(6):3088-3093.
51. Butt E, Abel K, Kriegerm M, Palm D, Hoppe V, Hoppe J, Walter U. cAMP- and cGMP-dependent protein kinase phosphorylation sites of the focal adhesion vasodilator-stimulated phosphoprotein (VASP) in vitro and in intact human platelets. J Biol Chem 1994; 269(20):14509-14517.
52. Aszodi A, Pfeifer A, Abmad M, Glauner M, Zhou X, Ny L, Andersson K, Kehrel B, Offermanns S, Fässler R the vasodilator-stimulated phosphoprotein (VASP) is involved in cGMP- and cAMP-mediated inhibition of agonist-induced platelet aggregation, but is dispensable for smooth muscle function. EMBO J. 1999; 18(1):37-48.
53. Nagy A. Cre recombinase: the universal reagent for genome tailoring. Genesis 2000; 26(2):99-109.
54. Regan C P, Manabe I, Owens G K Development of a smooth muscle-targeted cre recombinase mouse reveals novel insights regarding smooth muscle myosin heavy chain promoter regulation. Circ Res 2000; 87(5):363-369.
55. Feil R, Mascrez B B, Metzger D, Chambon P. Ligand-activated site-specific recombination in mice. PNAS 1996; 93:10887-10890.
56. Camus A, Mesbah K, Rallu M, Babinet C, Barra J. Gene trap insertion reveals two open reading frames in the mouse SSeCKS gene: the form predominantly detected in the nervous system is suppressed by the insertion while the other, specific of the testis, remains expressed. Mech Develop 2001; 105:79-91.
57. Piccin A, Salameh A, Benna C, Sandrelli F, Mazzota G, Zordan M, Rosato E, Kyriacou C P, Costa R. Efficient and heritable functional knock-out of an adult phenotype in Drosophila using a GAL4-driven hairpin RNA incorporating a heterologous spacer. Nucleic Acids Res 2001; 29(12):E55-5.
58. Kim M, Yuan X, Okumura S, Ishikawa F. Successful inactivation of endogenous Oct-3/4 and c-mos genes in mouse preimplantation embryos and oocytes using short interfering RNAs. Biochem Bioph Res Co 2002; 296(5):1372-1377.
59. Chomcynski P, Sacchi N. Single-step method of RNA isolation by acid guanidinium thiocyanata-phenol-chloroform extraction. Anal Biochem 1987; 162:156-159.
60. Miano J M, Olson E N. Expression of the smooth muscle cell calponin gene marks the early cardiac and smooth muscle cell lineages during mouse embryogenesis. J Biol Chem 1996; 271(7095):7103.
61. Miano J M, Carlson M J, Spencer J A, Misra R P. Serum response factor-dependent regulation of the smooth muscle calponin gene. J Biol Chem 2000; 275(9814):9822.
62. Kumar A, Lindner V. Remodeling with neointima formation in the mouse carotid artery after the cessation of blood flow. Arterioscler Throm Vas 1997; 17(2238):2244.
63. Miano J M, Kelly L A, Artacho C A, Nuckolls T A, Piantedosi R, Blaner W S. all-Trans-retinoic acid reduces neointimal formation and promotes favorable geometric remodeling of the rat carotid artery after balloon withdrawal injury. Circulation 1998; 98:1219-1227.

### H. SEQUENCES

1. Sequence of Representative U6-LoxP Constructs

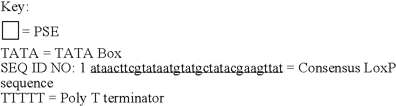

a) Representative PSE

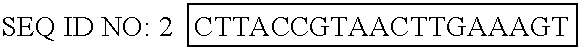

b) Representative Terminator

SEQ ID NO:3

TTTTTTCGTTTTTT or just TTTTT or some number of Ts

c) U6-LoxP Construct 1

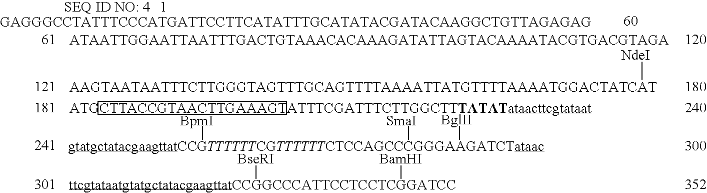

d) U6-LoxP Construct 2

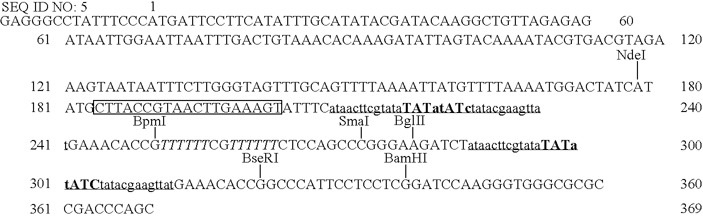

e) U6-LoxP Construct 3

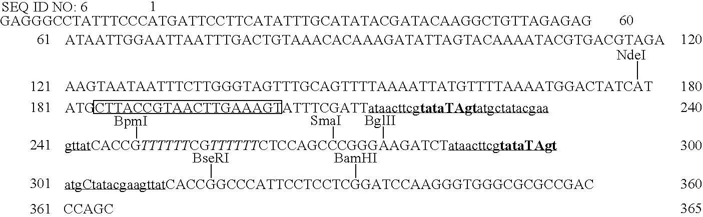

f) U6-LoxP Construct 4

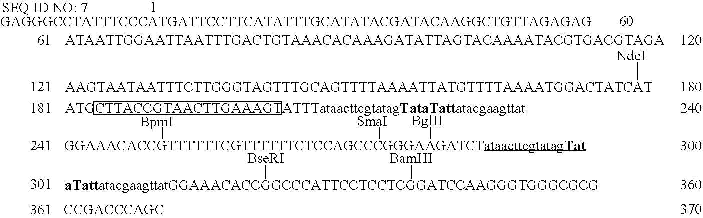

g) U6-mutantLoxP Construct

used to reduce interaction with Cre recombinase following recombination

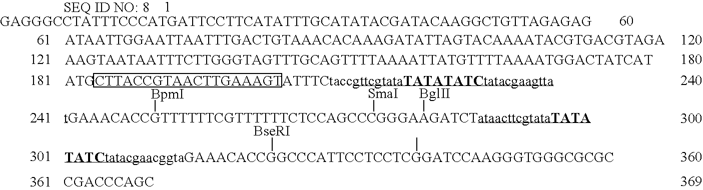

h) Construction of U6-LoxP Constructs

1. Replace U6 promoter with U6-LoxP promoter oligos ligated into NdeI-BseRI cut pSHAG or any other vector. It is understood that other restriction sites can be engineered and used to provide alternative cloning opportunities.

2. Insert linker oligo into BseRI-BamHI cut vector from #1.

i) SEQ ID NO. 9 X07425 Human gene for U 6 RNA

| | |
|---|---|
| 1 | aaggtcgggc aggaagaggg cctatttccc atgattcctt catatttgca tatacgatac |
| 61 | aaggctgtta gagagataat tagaattaat ttgactgtaa acacaaagat attagtacaa |
| 121 | aatacgtgac gtagaaagta ataatttctt gggtagtttg cagtttttaa aattatgttt |
| 181 | taaaatggac tatcatatgc ttaccgtaac ttgaaagtat ttcgatttct tggctttata |
| 241 | tatcttgtgg aaaggacgaa acaccgtgct cgcttcggca gcacatatac taaaattgga |
| 301 | acgatacaga gaagattagc atggcccctg cgcaaggatg acacgcaaat tcgtgaagcg |
| 361 | ttccatattt ttacatcagg ttgtttttct gtttttacat caggttgttt ttctgtttgg |
| 421 | tttttttttt acaccacgtt tatacgccgg tgcacggttt acca |

j) SEQ ID NO. 10 X06980 Mouse gene for U 6 RNA

| | |
|---|---|
| 1 | gatccgacgc cgccatctct aggcccgcgc cggccccctc gcacagactt gtgggagaag |
| 61 | ctcggctact cccctgcccc ggttaatttg catataatat ttcctagtaa ctatagaggc |
| 121 | ttaatgtgcg ataaaagaca gataatctgt tctttttaat actagctaca ttttacatga |
| 181 | taggcttgga tttctataag agatacaaat actaaattat tattttaaaa aacagcacaa |
| 241 | aaggaaactc accctaactg taaagtaatt gtgtgttttg agactataaa tatcccttgg |
| 301 | agaaaagcct tgtttgtgct cgcttcggca gcacatatac taaaattgga acgatacaga |
| 361 | gaagattagc atggcccctg cgcaaggatg acacgcaaat tcgtgaagcg ttccatattt |
| 421 | tgttcctcag aggaactgac aagcacccta acatcctatt ggaggctcac tcacgttttt |
| 481 | tctattttgt ttcttgacag cagagctcgt tgctcactgt atagctcagg ttggcctgac |
| 541 | actgatgagg ttctccagtg actgcctcta cctacctact gggatgacag aggtgtacca |
| 601 | ccaagccacg ggctcctgtg tgagtgtgtg tgtgtgtgta taagtgtgcc ttccacagtg |
| 661 | cacgtaagag gacaaggagt tggttcttgc tctcagatca tcaagct |

k) SEQ ID NO. 11 M63671 Mus musculus 7SK Class mRNA Gene

| | |
|---|---|
| 1 | tttaaactta gaacgaagcg agtataaaaa ggattattta accctaaaac ggattcagga |
| 61 | tttgttataa tatcaagtac agtcggctac ataaggtcac cacatgtgta aagttacaaa |
| 121 | attctatggc cttatatacc taccaagagc ctgagtactc tcggatgtga gggcgatctg |
| 181 | gctgcgacat ctgtcacccc attgatcgcc agggttgatt cggctgatct ggctggctag |
| 241 | gcgggtgtcc ccttcctccc tcaccgctcc atgtgcgtcc ctcccgaagc tgcgcgctcg |
| 301 | gtcgaagagg acgaccttcc ccgaatagag gaggaccggt cttcggtcaa gggtatacga |
| 361 | gtagctgcgc tcctctgcta gaacctccaa acaagctctc aaggtccatt gtaggagaac |
| 421 | gtagggtagt caagcttcca agactccaga cacatccaaa tgaggcgctg catgtggcag |
| 481 | tctgctttct tttgtagttc ctgcaattta attttcgttt aaa |

l) SEQ ID NO. 12 S68670 H1 RNA Gene {promoter}

| | |
|---|---|
| 1 | atttgcatgt cgctatgtgt tctgggaaat caccataaac gtgaaatgtc tttggatttg |
| 61 | ggaatcttat aagttctgta tgagaccact ctttcccata gggcggaggg aagctcatca |
| 121 | gtggggccac gagctgagtg cgtcctgtca ctccactccc atgtcccttg ggaaggtctg |
| 181 | agactagggc cagaggcggc cctaacaggg ctctccctga gcttcaggga ggtgagttcc |
| 241 | cagagaacgg ggctccgcgc gaggtcagac tgggcaggag atgccgtgga ccccgccctt |
| 301 | cggggagggg cccggcggat gcctcctttg ccggagcttg gaacagactc acggccagcg |
| 361 | aagtgagttc aatggctgag gtgaggtacc ccgcagggga cctcataacc caattcagac |
| 421 | cactctcctc cgcccatttt tggaaaaaaa aaaaaaaaaa aaaaacaaaa cgaaaccggg |
| 481 | ccgggcgcgg tggttca |

m) SEQ ID NO. 13 Human U6 Promoter

| | |
|---|---|
| 1 | aaggtcgggc aggaagaggg cctatttccc atgattcctt catatttgca tatacgatac |
| 61 | aaggctgtta gagagataat tagaattaat ttgactgtaa acacaaagat attagtacaa |
| 121 | aatacgtgac gtagaaagta ataatttctt gggtagtttg cagtttttaa aattatgttt |
| 181 | taaaatggac tatcatatgc ttaccgtaac ttgaaagtat ttcgatttct tggctttata |
| 241 | tatcttgtgg aaaggacgaa acaccg |

n) SEQ ID NO. 14 Human H1 Promoter

| | |
|---|---|
| 1 | ttatagggag ctgaagggaa gggggtcaca gtaggtggca tcgttccttt ctgactgccc |
| 61 | gccccccgca tgccgtcccg cgatattgag ctccgaacct ctcgccctgc cgccgccggt |
| 121 | gctccgtcgc cgccgcgccg ccatggaatt cgaacgctga cgtcatcaac ccgctccaag |
| 181 | gaatcgcggg cccagtgtca ctaggcggga acacccagcg cgcgtgcgcc ctggcaggaa |
| 241 | gatggctgtg agggacaggg gagtggcgcc ctgcaatatt tgcatgtcgc tatgtgttct |
| 301 | gggaaatcac cataaacgtg aaatgtcttt ggatttggga atcttataag ttctgtatga |
| 361 | gaccactctt tccc |

What is claimed is:

1. A nucleic acid comprising a promoter and a set of recombinase sites, comprising a first recombination site and a second recombination site, wherein the promoter and the set of recombinase sites are oriented so that transcription from a transcription start site is increased when a recombinase acts on the set of recombinase sites after the nucleic acid is attached to a second nucleic acid forming a third nucleic acid and wherein the third nucleic acid comprises a transcription start site.

2. The nucleic acid of claim 1, further comprising a decoy sequence in between the recombinase sites

3. The nucleic acid of claim 2, wherein the decoy sequence comprises a marker sequence.

4. The nucleic acid of claim 3, wherein the marker sequence confers antibiotic resistance

5. The nucleic acid of claim 3, wherein the marker sequence confers Neomycin Resistance, Zeocin Resistance, Blasticidin Resistance, or Hygromycin Resistance.

6. The nucleic acid of claim 3, wherein the marker sequence is a fluorescent protein.

7. The nucleic acid of claim 3, wherein the marker sequence is Beta-Galactosidase, or Green Fluorescent Protein. EGFP, ECFP, EYFP, HcRed or DsRed

8. The nucleic acid of claim 2, wherein the decoy comprises a transcription inhibitor recognition site.

9. The nucleic acid of claim 8, wherein the transcription inhibitor site is TTTTT or SEQ ID NO:3.

10. The nucleic acid of claim 1, further comprising a transgene.

11. The nucleic acid of claim 10, wherein the transgene comprises a functional nucleic acid, a sequence encoding a protein, or a marker.

12. The nucleic acid of claim 10, wherein the transgene comprises a functional nucleic acid.

13. The nucleic acid of claim 12, wherein the functional nucleic comprises an antisense molecule, a ribozymes, an external guide sequence, a triple helix forming nucleic acid, or an RNAi nucleic acid.

14. The nucleic acid of claim 1, wherein the promoter is an RNA polymerase III promoter.

15. The nucleic acid of claim 14, wherein the RNA polymerase III promoter comprises a sequence having at least 80% identity to a sequence set forth in SEQ ID NO: 2

16. The nucleic acid of claim 15, wherein the RNA polymerase III promoter comprises a sequence having the sequence set forth in SEQ ID NO:2.

17. The nucleic acid of claim 14, wherein the RNA polymerase III promoter comprises a type 1, type 2, or type 3 polymerase III promoter.

18. The nucleic acid of claim 15, wherein the RNA polymerase III promoter comprises a U6, 7SK, hY4, hY5 and H1.

19. The nucleic acid of claim 18, wherein the RNA polymerase III promoter comprises a sequence set forth in SEQ ID NO: 9-14

20. The nucleic acid of claim 1, wherein the recombinase sites are recognized by a lambda integrase.

21. The nucleic acid of claim 20, wherein the lambda integrase comprises phage lambda integrase, bacteriophage P1 Cre recombinase, a XerC/XerD recombinases, or Flp recombinase.

22. The nucleic acid of claim 1, wherein the recombinase sites are recognized by a resolvase-invertase.

23. The nucleic acid of claim 22, wherein the resolvase-invertase comprises a gamma-delta resolvase, TN3 transposon resolvase, Gin invertase, or a Hin invertase.

24. The nucleic acid of claim 1, wherein the promoter and the set of recombinase sites are oriented 5′ to 3′ promoter then first recombinase site then second recombinase site.

25. The nucleic acid of claim 1, further comprising a marker sequence or transgene.

26. A vector comprising the nucleic acid of claim 1.

27. A cell comprising the nucleic acid of claim 1.

28. An non-human organism comprising the nucleic acid of claim 1.

29. A non human mammal comprising the nucleic acid of claim 1.

30. A nucleic acid comprising a promoter and one member of recombinase set, wherein the promoter and the recombinase site are oriented so that when the nucleic acid is attached to a second nucleic acid comprising a transcription start site and a second member of the recombinase set transcription from the transcription start site is increased when a recombinase acts on the recombinase sets.

31. A nucleic acid comprising a promoter, a set of recombinase sites, and a transcription start site.

32. The nucleic acid of claim 1, wherein the nucleic acid is SEQ ID NO:3, SEQ ID NO:4, SEQ ID NO:5, SEQ ID NO:6, SEQ ID NO:7, or SEQ ID NO:8.

33. The nucleic acid of claim 1, wherein the promoter, the set of recombinase sites, and the transcription start site are oriented so that when the nucleic acid is attached to a second nucleic acid comprising a transcription start site, transcription from the transcription start site is increased when a recombinase acts on the set of recombinase sites.

34. A nucleic acid comprising a promoter and a set of recombinase sites, comprising a first recombination site and a second recombination site, wherein the promoter and the set of recombinase sites are oriented so that transcription from a transcription start site is repressed before a recombinase acts on the set of recombinase sites, after the nucleic acid is attached to a second nucleic acid forming a third nucleic acid and wherein the third nucleic acid comprises a transcription start site.
